SEQUENCE LISTING (15 sequences)

SEQ ID NO: 1 - protein, 535 aa, Artificial Sequence
  CRM197
GADDVVDSSKSFVMENFSSYHGTKPGYVDSIQKGIQKPKSGTQGNYDDDWKEFYSTDNKYDAAGYSVDNENPLSGKAGGVVKVTYPGLTKVLALKVDNAETIKKELGLSLTEPLMEQVGTEEFIKRFGDGASRVVLSLPFAEGSSSVEYINNWEQAKALSVELEINFETRGKRGQDAMYEYMAQACAGNRVRRSVGSSLSCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPAYSPGHKTQPFLHDGYAVSWNTVEDSIIRTGFQGESGHDIKITAENTPLPIAGVLLPTIPGKLDVNKSKTHISVNGRKIRMRCRAIDGDVTFCRPKSPVYVGNGVHANLHVAFHRSSSEKIHSNEISSDSIGVLGYQKTVDHTKVNSKLSLFFEIKS

SEQ ID NO: 2 - protein, 428 aa, Artificial Sequence
  hIgE constant region
ASTQSPSVFPLTRCCKNIPSNATSVTLGCLATGYFPEPVMVTWDTGSLNGTTMTLPATTLTLSGHYATISLLTVSGAWAKQMFTCRVAHTPSSTDWVDNKTFSVCSRDFTPPTVKILQSSCDGGGHFPPTIQLLCLVSGYTPGTINITWLEDGQVMDVDLSTASTTQEGELASTQSELTLSQKHWLSDRTYTCQVTYQGHTFEDSTKKCADSNPRGVSAYLSRPSPFDLFIRKSPTITCLVVDLAPSKGTVNLTWSRASGKPVNHSTRKEEKQRNGTLTVTSTLPVGTRDWIEGETYQCRVTHPHLPRALMRSTTKTSGPRAAPEVYAFATPEWPGSRDKRTLACLIQNFMPEDISVQWLHNEVQLPDARHSTTQPRKTKGSGFFVFSRLEVTRAEWEQKDEFICRAVHEAASPSQTVQRAVSVNPGK

SEQ ID NO: 3 - protein, 105 aa, Artificial Sequence
  hIgE CN53
PRGVSAYLSRPSPFDLFIRKSPTITCLVVDLAPSKGTVNLTWSRASGKPVNHSTRKEEKQRNGTLTVTSTLPVGTRDWIEGETYQCRVTHPHLPRALMRSTTKTS

SEQ ID NO: 4 - protein, 210 aa, Artificial Sequence
  hIgE CN53 and CN54
PRGVSAYLSRPSPFDLFIRKSPTITCLVVDLAPSKGTVNLTWSRASGKPVNHSTRKEEKQRNGTLTVTSTLPVGTRDWIEGETYQCRVTHPHLPRALMRSTTKTSGPRAAPEVYAFATPEWPGSRDKRTLACLIQNFMPEDISVQWLHNEVQLPDARHSTTQPRKTKGSGFFVFSRLEVTRAEWEQKDEFICRAVHEAASPSQTVQRAVS

SEQ ID NO: 5 - protein, 207 aa, Artificial Sequence
  hIgE CN52 and CN53
PTVKILQSSCDGGGHFPPTIQLLCLVSGYTPGTINITWLEDGQVMDVDLSTASTTQEGELASTQSELTLSQKHWLSDRTYTCQVTYQGHTFEDSTKKCADSNPRGVSAYLSRPSPFDLFIRKSPTITCLVVDLAPSKGTVNLTWSRASGKPVNHSTRKEEKQRNGTLTVTSTLPVGTRDWIEGETYQCRVTHPHLPRALMRSTTKTS

SEQ ID NO: 6 - protein, 312 aa, Artificial Sequence
  hIgE CN52, CN53 and CN54
PTVKILQSSCDGGGHFPPTIQLLCLVSGYTPGTINITWLEDGQVMDVDLSTASTTQEGELASTQSELTLSQKHWLSDRTYTCQVTYQGHTFEDSTKKCADSNPRGVSAYLSRPSPFDLFIRKSPTITCLVVDLAPSKGTVNLTWSRASGKPVNHSTRKEEKQRNGTLTVTSTLPVGTRDWIEGETYQCRVTHPHLPRALMRSTTKTSGPRAAPEVYAFATPEWPGSRDKRTLACLIQNFMPEDISVQWLHNEVQLPDARHSTTQPRKTKGSGFFVFSRLEVTRAEWEQKDEFICRAVHEAASPSQTVQRAVS

SEQ ID NO: 7 - protein, 219 aa, Artificial Sequence
  peptide sequence + G335C
ADSNPRCVSAYLSRPSPFDLFIRKSPTITCLVVDLAPSKGTVNLTWSRASGKPVNHSTRKEEKQRNGTLTVTSTLPVGTRDWIEGETYQCRVTHPHLPRALMRSTTKTSGPRAAPEVYAFATPEWPGSRDKRTLACLIQNFMPEDISVQWLHNEVQLPDARHSTTQPRKTKGSGFFVFSRLEVTRAEWEQKDEFICRAVHEAASPSQTVQRAVSVNPGK

SEQ ID NO: 8 - protein, 105 aa, Artificial Sequence
  hIgE CN53 + G335C
PRCVSAYLSRPSPFDLFIRKSPTITCLVVDLAPSKGTVNLTWSRASGKPVNHSTRKEEKQRNGTLTVTSTLPVGTRDWIEGETYQCRVTHPHLPRALMRSTTKTS

SEQ ID NO: 9 - protein, 210 aa, Artificial Sequence
  hIgE CN53 and CN54 + G335C
PRCVSAYLSRPSPFDLFIRKSPTITCLVVDLAPSKGTVNLTWSRASGKPVNHSTRKEEKQRNGTLTVTSTLPVGTRDWIEGETYQCRVTHPHLPRALMRSTTKTSGPRAAPEVYAFATPEWPGSRDKRTLACLIQNFMPEDISVQWLHNEVQLPDARHSTTQPRKTKGSGFFVFSRLEVTRAEWEQKDEFICRAVHEAASPSQTVQRAVS

SEQ ID NO: 10 - protein, 207 aa, Artificial Sequence
  hIgE CN52 and CN53 + G335C
PTVKILQSSCDGGGHFPPTIQLLCLVSGYTPGTINITWLEDGQVMDVDLSTASTTQEGELASTQSELTLSQKHWLSDRTYTCQVTYQGHTFEDSTKKCADSNPRCVSAYLSRPSPFDLFIRKSPTITCLVVDLAPSKGTVNLTWSRASGKPVNHSTRKEEKQRNGTLTVTSTLPVGTRDWIEGETYQCRVTHPHLPRALMRSTTKTS

SEQ ID NO: 11 - protein, 312 aa, Artificial Sequence
  hIgE CN52, CN53 and CN54 + G335C
PTVKILQSSCDGGGHFPPTIQLLCLVSGYTPGTINITWLEDGQVMDVDLSTASTTQEGELASTQSELTLSQKHWLSDRTYTCQVTYQGHTFEDSTKKCADSNPRCVSAYLSRPSPFDLFIRKSPTITCLVVDLAPSKGTVNLTWSRASGKPVNHSTRKEEKQRNGTLTVTSTLPVGTRDWIEGETYQCRVTHPHLPRALMRSTTKTSGPRAAPEVYAFATPEWPGSRDKRTLACLIQNFMPEDISVQWLHNEVQLPDARHSTTQPRKTKGSGFFVFSRLEVTRAEWEQKDEFICRAVHEAASPSQTVQRAVS

SEQ ID NO: 12 - protein, 219 aa, Artificial Sequence
  peptide sequence
ADSNPRGVSAYLSRPSPFDLFIRKSPTITCLVVDLAPSKGTVNLTWSRASGKPVNHSTRKEEKQRNGTLTVTSTLPVGTRDWIEGETYQCRVTHPHLPRALMRSTTKTSGPRAAPEVYAFATPEWPGSRDKRTLACLIQNFMPEDISVQWLHNEVQLPDARHSTTQPRKTKGSGFFVFSRLEVTRAEWEQKDEFICRAVHEAASPSQTVQRAVSVNPGK

SEQ ID NO: 13 - protein, 419 aa, Artificial Sequence
  EIgE constant domain
VSKQAPLIFPLAACCKDTKTTNITLGCLVKGYFPGAWDAGPLNPSTMTFPAVFDQTSGLYTTISRVVASGKWAKQKFTCGVVHSQETFNKTFNACIVTFTPPTVKLFHSSCDPGGDSHTTIQLLCLISDYTPGDIDIVWLIEGQKVDEQFPTQASMKQEGSWPPTHSELNINQGQWASENTYTCQVTYKDMIFNQARKCTESDPPGVSVYLSPPSPLDLYVSKTPKITCLVVDLANVQGLSLNWSRESGEPLQKHTLATSEQFNKTFSVTSTLPVDTTDWIEGETYKCTVSHPDLPREVVRSIAKAPGKRLSPEVYVFLPPEEDQSSKDKVTLTCLIQNFFPADISVQWRRNNVLIQTDQQATTRPQKANGPDPAFFVFSRLEVSRAEWEQKNKFACKVVHEALSQRTLQKEVSKDPGK

SEQ ID NO: 14 - protein, 426 aa, Artificial Sequence
  cIgE constant domain
TSQDLSVFPLASCCKDNIASTSVTLGCLVTGYLPMSTTVTWDTGSLNKNVTTFPTTFHETYGLHSIVSQVTASGKWAKQRFTCSVAHAESTAINKTFSACALNFIPPTVKLFHSSCNPVGDTHTTIQLLCLISGYVPGDMEVIWLVDGQKATNIFPYTAPGTKEGNVTSTHSELNITQGEWVSQKTYTCQVTYQGFTFKDEARKCSESDPRGVTSYLSPPSPLDLYVHKAPKITCLVVDLATMEGMNLTWYRESKEPVNPGPLNKKDHFNGTITVTSTLPVNTNDWIEGETYYCRVTHPHLPKDIVRSIAKAPGKRAPPDVYLFLPPEEEQGTKDRVTLTCLIQNFFPADISVQWLRNDSPIQTDQYTTTGPHKVSGSRPAFFIFSRLEVSRVDWEQKNKFTCQVVHEALSGSRILQKWVSKTPGK

SEQ ID NO: 15 - protein, 496 aa, Artificial Sequence
  fIgE constant domain
AYISSGGNTDYADSVKGRFSISRDNAKNTLYLQMTSLKTEDTATYYCARGTGVIPDYWGQGALVTVSSTSIQAPLVFPLATCCKGTIATAPSVTLGCLVTGYFPMPVTVTWDARSLNKSVVTLPATLQENSGLYTTTSHVTVSGEWAKQKFTCSVAHAESPTINKTVSACTMNFIPPTVKLFHSSCNPLGDTGSTIQLLCLISGYVPGDMEVTWLVDGQKATNIFPYTAPGKQEGKVTSTHSELNITQGEWVSQKTYTCQVTYQGFTFEDHARKCTESDPRGVSTYLSPPSPLDLYVHKSPKITCLVVDLANTDGMILTWSRENGESVHPDPMVKKTQYNGTITVTSTLPVDATDWVEGETYQCKVTHPDLPKDIVRSIAKAPGRRFPPEVYVFLPPEGEPKTKDKVTLTCLIQNFFPPDISVQWLHNDSPVRTEQQATTWPHKATGPSPAFFVFSRLEVSRADWEQRDVFTCQVVHEALPGFRTLKKSVSKNPGK

(19) United States
(12) Patent Application Publication
(10) Pub. No.: US 2023/0364232 A1
(43) Pub. Date: Nov. 16, 2023

(54) IMMUNOGENIC PRODUCT COMPRISING AN IgE FRAGMENT FOR TREATING IgE-MEDIATED INFLAMMATORY DISORDERS

(71) Applicants: NEOVACS, Paris (FR); INSERM (INSTITUT NATIONAL DE LA SANTÉ ET DE LA RECHERCHE MÉDICALE), Paris (FR); INSTITUT PASTEUR, Paris (FR)

(72) Inventors: Laurent REBER, Tournefeuille (FR); Pierre BRUHNS, PARIS (FR); Eva CONDE GARCIA, PARIS (FR); Marija BACKOVIC, PARIS (FR); Vincent SERRA, Bondoufle (FR); Géraldine GROUARD-VOGEL, PARIS (FR); Romain BERTRAND, ZÜRICH (CH)

(73) Assignees: NEOVACS, Paris (FR); INSERM (INSTITUT NATIONAL DE LA SANTÉ ET DE LA RECHERCHE MÉDICALE), Paris (FR); INSTITUT PASTEUR, Paris (FR)

(21) Appl. No.: 18/245,239

(22) PCT Filed: Sep. 17, 2021
(86) PCT No.: PCT/EP2021/075732

§ 371 Date: Mar. 14, 2023

Related U.S. Application Data
(60) Provisional application No. 63/079,686, filed on Sep. 17, 2020.

(30) Foreign Application Priority Data
Sep. 17, 2020 (EP) 20306047.0

(51) Int. Cl. A61K 39/44; A61K 39/35; A61P 37/08

(52) U.S. Cl. CPC A61K 39/44; A61K 39/35; A61P 37/08; A61K 2039/6037

(57) ABSTRACT

An immunogenic product including at least one immunoglobulin or fragment thereof conjugated with a carrier protein, wherein the at least one immunoglobulin is IgE and preferably wherein the IgE fragment includes the IgE Cε3 domain, and wherein the carrier protein is preferably CRM₁₉₇. Also the use of this immunogenic product for treating inflammatory disorders, and in particular allergic disorders.

### FIELD OF INVENTION

The present invention relates to an immunogenic product and to the use thereof for treating disorders associated with aberrant IgE expression or activity, in particular IgE-mediated allergies such as food and venom allergies and anaphylaxis.

### BACKGROUND OF INVENTION

The prevalence of allergic diseases has dramatically increased over the past years, especially in industrialized countries, with more than 30% of children suffering from allergies. The most dramatic clinical manifestation of allergy is anaphylaxis, an acute and potentially fatal systemic reaction Immunoglobulin E (IgE) plays a central role in mediating allergic reactions and anaphylaxis. Upon exposure to an allergen, such allergen is recognized by allergen-specific IgE bound to their high-affinity receptor FcεRI on the surface of tissue mast cells and blood basophils, which promotes the degranulation of these cells, and the release of both preformed and newly synthesized mediators, including histamine. For this reason, clinical diagnoses of allergies are largely based on measurements of allergen-specific IgE.

Most treatments for allergies are symptomatic (mostly antihistamines and corticosteroids). In recent years however, several recombinant monoclonal antibodies (mAbs) have been developed for the treatment of allergies. Omalizumab, a humanized anti-IgE mAb, shows clinical benefit for the treatment of allergic asthma and chronic spontaneous urticaria. Available clinical data suggest that this mAb could also have benefit for the treatment of other types of allergies including food allergy. However, use of omalizumab (or any other mAb) is limited first and foremost by high cost and the need to perform repeated injections, and also by potential risks of appearance of anti-drug antibodies (ADAs) or other adverse reactions. The main medical limitation is patients with levels of IgE higher than 700 IU/ml, that may be of risk of anaphylaxis if treated with omalizumab. A next-generation of anti-IgE mAb, ligelizumab, with significantly higher affinity for IgE than omalizumab and potentially reduced adverse effects, has been developed but did not demonstrate improved efficacy over omalizumab in severe asthma patients (NCT02075008). Omalizumab and ligelizumab differ in the epitopes they bind on IgE and on their ability to interfere with FcεRI-bound IgE or IgE production.

Therefore, while IgE are promising therapeutic targets for the treatment of allergies and anaphylaxis, there is a clear need to improve current strategies to block IgE, in order to reach long-term therapeutic effects.

Therapeutic conjugate vaccines called kinoids are used in an active immunization strategy to induce neutralizing antibodies against an abnormally highly produced target, to reduce target levels back to baseline or lower.

Several approaches based on such therapeutic conjugate have been developed for the prevention and treatment of IgE-related disorders, including the generation of immunogens comprising antigenic peptides of IgE linked to an immunogenic carrier (WO2011/154878, WO2010/067286, WO99/67293, Peng et al., 2007, Spiegelberg et al., 1987). However, so far, none of these conjugates (comprising only small peptides of IgE) was therapeutically validated. In particular, experimental results obtained during a phase I clinical study showed that peptides derived from IgE and coupled to a carrier did not lead to significant lowering of serum IgE in the majority of subjects. Consequently, there is still a need for compounds efficient for inducing neutralizing antibodies against IgE in patients in need thereof.

In the present invention, the Applicants generated an anti-human IgE kinoid comprising the Cε3 domain of human IgE coupled to the non-toxic mutant of diphtheria toxin, CRM₁₉₇. This vaccine induced a long-lasting anti-human IgE neutralizing antibody response without any adverse effects in mice humanized for both IgE and FcεRI (IgE/FcεRI humanized mice). Anti-IgE vaccination reduced both circulating IgE levels and levels of IgE bound to their high-affinity receptor FcεRI at the surface of blood basophils, and fully protected against IgE-mediated anaphylaxis in IgE/FcεRI humanized mice.

### SUMMARY

The present invention relates to an immunogenic product comprising at least one immunoglobulin or immunoglobulin fragment conjugated with a carrier protein, wherein the at least one immunoglobulin is IgE, preferably human IgE, and wherein the IgE fragment comprises the IgE Cε3 domain, and wherein the carrier protein is preferably CRM₁₉₇.

In one embodiment, the immunoglobulin fragment comprises a part or the totality of the IgE Cε3 and Cε4 domains.

In one embodiment, the immunoglobulin fragment comprises a part or the totality of the IgE Cε2, Cε3 and Cε4 domains.

In one embodiment, the IgE or the fragment thereof comprises the G335C mutation.

In one embodiment, the IgE fragment comprises or consists in SEQ ID NO:7.

In one embodiment, the IgE fragment comprises at least one glycosylation.

Another object of the present invention is a composition comprising the immunogenic product as described hereinabove.

Another object of the present invention is a pharmaceutical composition comprising the immunogenic product as described hereinabove and at least one pharmaceutically acceptable excipient.

Another object of the present invention is a vaccine composition comprising the immunogenic product as described hereinabove and at least one adjuvant.

In one embodiment, the composition, pharmaceutical composition or vaccine composition as described hereinabove is an emulsion.

The present invention further relates to a method for producing an immunogenic product as described hereinabove, the method comprising steps of:
a) contacting the immunoglobulin or fragment thereof with a heterobifunctional crosslinker containing a NHS-ester, preferably N-[γ-maleimidobutyryloxy]-succinimide ester (sGMBS), thereby obtaining a complex between a heterobifunctional crosslinker containing a NHS-ester and the immunoglobulin or fragment thereof, preferably a sGMBS-immunoglobulin or fragment thereof complex;
b) contacting the carrier protein with a heterobifunctional crosslinker containing a NHS-ester, preferably N-succinimidyl-S-acetylthioacetate (SATA) to generate a complex between the heterobifunctional crosslinker containing a NHS-ester and the carrier, preferably carrier-SATA complex;
c) contacting the complex between a heterobifunctional crosslinker containing a NHS-ester and the immunoglobulin or fragment thereof, preferably the sGMBS-immunoglobulin or fragment thereof complex obtained at step (a) with the complex between the heterobifunctional crosslinker containing a NHS-ester and the carrier, preferably the carrier carrier-SATA complex obtained at step (b).

Another object of the present invention is an immunogenic product as described herein for use as a medicament.

The present invention further relates to an immunogenic product or a composition, pharmaceutical composition or vaccine composition as described herein, for treating an inflammatory disorder, preferably wherein the inflammatory disorder is associated with aberrant IgE expression or activity.

In one embodiment, the inflammatory disorder is selected from the group comprising asthma, allergic conditions (such as, for example, food allergies, venom allergy, allergy to animals, drug allergy, hyper IgE syndrome, allergic rhinitis, allergic conjunctivitis and allergic enterogastritis), anaphylaxis, atopic disorders (such as, for example, urticaria (including chronic idiopathic urticaria and chronic spontaneous urticaria), eczema), bullous pemphigoid, respiratory disorders (such as asthma, allergic bronchopulmonary aspergillosis, allergic bronchopulmonary mycosis), nasal polyposis and other conditions involving airway inflammation (such as, for example, eosinophilia, fibrosis and excess mucus production including cystic fibrosis, systemic sclerosis (SSc)); inflammatory and/or autoimmune disorders or conditions, gastrointestinal disorders or conditions (such as, for example, inflammatory bowel diseases (IBD) and eosinophilic esophagitis (EE), and eosinophilic-mediated gastrointestinal disease, ulcerative colitis and Crohn's disease); systemic lupus erythematosus; mastocytosis and mast cell activation syndrome (MCAS).

In one embodiment, the inflammatory disorder is selected from allergy, anaphylaxis, allergic asthma, allergic rhinitis, allergic conjunctivitis, nasal polyposis, preferably said inflammatory disorder is food or venom allergy.

The present invention further relates to an immunogenic product or a composition, pharmaceutical composition or vaccine composition as described hereinabove for inducing desensitization of a subject allergic to a specific antigen, wherein said immunogenic product or composition and said specific antigen are to be administered to the allergic subject.

### Definitions

In the present invention, the following terms have the following meanings:
As used herein, the term “about” when referring to a measurable value such as an amount, a temporal duration, and the like, is meant to encompass variations of ±20% or in some instances±10%, or in some instances±5%, or in some instances±1%, or in some instances±0.1% from the specified value, as such variations are appropriate to perform the disclosed methods.
As used herein, an “adjuvant” is a substance that enhances the immunogenicity of an immunogenic product of this invention. Adjuvants are often given to boost the immune response and are well known to the skilled artisan.
As used herein, the term “carrier protein molecule” refers to a protein or a peptide of at least 15, 30 or 50 amino acids long which is immunogenic when injected to a subject (e.g., a human, a cat, a dog or a horse) and which, when it is partially covalently associated to at least one IgE or fragment thereof (wherein preferably the IgE fragment comprises the IgE Cε3 domain) for forming heterocomplexes, allows for a large number of antigens of said at least one IgE or fragment thereof to be presented to the B lymphocytes, and for subsequent production of antibodies directed against IgE or fragment thereof.
As used herein, the term “immune response” refers to the action, for example of lymphocytes, antigen presenting cells, phagocytic cells and macromolecules produced by the above cells or the liver (including antibodies, cytokines and complement).
As used herein, the term “immunogenic product” refers to at least one IgE or fragment thereof coupled to a carrier protein that induces an immune response in a subject, preferably a mammal, to whom said immunogenic product is administered, including a humoral immune response, i.e., the production of antibodies that neutralize the properties, such as, for example, the biological activity of endogenous IgE.
As used herein, the term “pharmaceutically acceptable excipient” refers to an excipient that does not produce an adverse, allergic or other untoward reaction when administered to a mammal, preferably a human. It includes any and all solvents, dispersion media, coatings, antibacterial and antifungal agents, isotonic and absorption delaying agents and the like. A pharmaceutically acceptable carrier or excipient may thus refer to a non-toxic solid, semi-solid or liquid filler, diluent, encapsulating material or formulation auxiliary of any type. For human administration, preparations should meet sterility, pyrogenicity, general safety and purity standards as required by the regulatory offices such as the FDA or EMA.
As used herein, the term “recombinant protein” refers to a protein (e.g., IgE or a fragment thereof or a carrier protein) which is generated using recombinant DNA technology, such as, for example, a protein (e.g., IgE or a fragment thereof or a carrier protein) expressed in prokaryote cells (using a bacteriophage or a plasmid expression system) or in eukaryotic cells (such as for example yeast, insect or mammalian expression system). This term should also be construed to mean a protein (e.g., IgE or a fragment thereof or a carrier protein) which has been generated by the synthesis of a DNA molecule encoding the protein (e.g., IgE or a fragment thereof or a carrier protein) and which DNA molecule expresses a protein (e.g., IgE or a fragment thereof or a carrier protein), or an amino acid sequence specifying the protein (e.g., IgE or a fragment thereof or a carrier protein), wherein the DNA or amino acid sequence has been obtained using recombinant DNA or amino acid sequence technology which is available and well known in the art.
As used herein, the term “subject” is intended to include living organisms in which an immune response can be elicited (e.g., mammals, in particular human, primates, dogs, cats, horses, sheep and the like). Preferably, the subject is a human. In one embodiment, a subject may be a “patient”, i.e., a warm-blooded animal, preferably a human, who/which is awaiting the receipt of, or is receiving medical care or was/is/will be the object of a medical procedure or is monitored for the development of the targeted disease or condition, such as, for example, an inflammatory disorder. In one embodiment, the subject is an adult (for example a subject above the age of 18). In another embodiment, the subject is a child (for example a subject below the age of 18). In one embodiment, the subject is a male. In another embodiment, the subject is a female. In one embodiment, the subject is affected, preferably is diagnosed, with an inflammatory disorder. In one embodiment, the subject is at risk of developing an inflammatory disorder. Examples of risks factor include, but are not limited to, genetic predisposition, or familial history of inflammatory disorders.
As used herein, the term “therapeutically effective amount” refers to an amount of the immunogenic product as described herein, effective to achieve a particular biological result. Thus, the term “therapeutically effective amount” means a level or amount of immunogenic product that is aimed at, without causing significant negative or adverse side effects to the target, (1) delaying or preventing the onset of the targeted disease or condition; (2) slowing down or stopping the progression, aggravation, or deterioration of one or more symptoms of the targeted disease or condition; (3) bringing about ameliorations of the symptoms of the targeted disease or condition; (4) reducing the severity or incidence of the targeted disease or condition; or (5) curing the targeted disease or condition. A therapeutically effective amount may be administered prior to the onset of the targeted disease or condition, for a prophylactic or preventive action. Alternatively, or additionally, the therapeutically effective amount may be administered after initiation of the targeted disease or condition, for a therapeutic action.
As used herein, the term “treatment” or “treating” refers to both therapeutic treatment and prophylactic or preventative measures; wherein the object is to prevent or slow down (lessen) the targeted disease or condition. Those in need of treatment include those already with the condition as well as those prone to have the condition or those in whom the condition is to be prevented. A subject is successfully “treated” for a disease or condition if, after receiving a therapeutic amount of an immunogenic product as described herein, the subject shows observable and/or measurable improvement in one or more of the following: reduction in the number of pathogenic cells; reduction in the percent of total cells that are pathogenic; relief to some extent of one or more of the symptoms associated with the specific condition; reduced morbidity and mortality, and/or improvement in quality of life issues. The above parameters for assessing successful treatment and improvement in the condition are readily measurable by routine procedures familiar to a physician.

### DETAILED DESCRIPTION

The present invention relates to an immunogenic product comprising at least one immunoglobulin or immunoglobulin fragment conjugated with at least one carrier protein, wherein the at least one immunoglobulin is an IgE.

The Applicants demonstrated that the administration of the immunogenic product of the present invention induces anti-IgE neutralizing antibodies in an animal model, thereby treating IgE-mediated inflammatory disorders.

Without willing to be bound to any theory, the Applicants suggest that the immunogenic product of the invention presents the advantage, as compared to the peptides conjugated to a carrier protein described in the art, to induce polyclonal anti-IgE neutralizing antibodies (i.e., antibodies that are directed to different epitopes on IgE sequence).

In one embodiment, the IgE fragment comprises or consists of the IgE Cε3 domain.

Examples of carrier proteins include, but are not limited to, CRM₁₉₇, KLH (Keyhole limpet hemocyanin), ovalbumin, bovine serum albumin (BSA), tetanus toxoid, diptheria toxoid, cholera toxoid, Neisseria meningitidis outer membrane protein in outer membrane vesicles, non-typeable Haemophilus influenza outer membrane protein, Pseudomonas aeruginosa toxin A and virus like particle (VLP).

In one embodiment, the carrier protein is CRM₁₉₇.

CRM₁₉₇ is a non-toxic mutant of diphtheria toxin having the sequence SEQ ID NO: 1, without toxic activity due to a single base substitution (mutation from glycine to glutamate in position 52).

SEQ ID NO: 1
GADDVVDSSKSFVMENFSSYHGTKPGYVDSIQKGIQKPKSGTQGNYDDD
WKEFYSTDNKYDAAGYSVDNENPLSGKAGGVVKVTYPGLTKVLALKVDN
AETIKKELGLSLTEPLMEQVGTEEFIKRFGDGASRVVLSLPFAEGSSSV
EYINNWEQAKALSVELEINFETRGKRGQDAMYEYMAQACAGNRVRRSVG
SSLSCINLDWDVIRDKTKTKIESLKEHGPIKNKMSESPNKTVSEEKAKQ
YLEEFHQTALEHPELSELKTVTGTNPVFAGANYAAWAVNVAQVIDSETA
DNLEKTTAALSILPGIGSVMGIADGAVHHNTEEIVAQSIALSSLMVAQA
IPLVGELVDIGFAAYNFVESIINLFQVVHNSYNRPAYSPGHKTQPFLHD
GYAVSWNTVEDSIIRTGFQGESGHDIKITAENTPLPIAGVLLPTIPGKL
DVNKSKTHISVNGRKIRMRCRAIDGDVTFCRPKSPVYVGNGVHANLHVA
FHRSSSEKIHSNEISSDSIGVLGYQKTVDHTKVNSKLSLFFEIKS

In one embodiment, CRM₁₉₇ may be obtained by conventional methods known in the art in autologous (C. diphtheriae) or heterologous systems (E. coli and P. fluorescens) as described by Hickey in 2018 (Hickey et al., 2018). For example, recombinant CRM₁₉₇ may be obtained by culturing cells containing an expression vector comprising a nucleic acid sequence (e.g., the gene) encoding CRM₁₉₇, harvesting inclusion bodies and purifying CRM₁₉₇. CRM₁₉₇ could also be extracted from culture of Corynebacterium diphtheriae from bacteria strain purchased at ATCC (ATCC39255). In one embodiment, CRM₁₉₇ is commercially available, and may be purchased, for example, from Reagent Proteins (San Diego, CA, US).

In one embodiment, the immunogenic product of the invention comprises a variant of CRM₁₉₇, wherein said variant presents at least about 70%, 75, 80, 85, 90, 95% or more identity with SEQ ID NO: 1. In one embodiment, said variant of CRM₁₉₇ comprises the mutation from glycine to glutamate in position 52 of CRM₁₉₇ and is thus non-toxic.

The term “identity” or “identical”, when used in a relationship between the sequences of two or more nucleic acid sequences or of two or more polypeptides, refers to the degree of sequence relatedness between nucleic acid sequences or polypeptides, as determined by the number of matches between strings of two or more nucleic or amino acid residues, respectively. “Identity” measures the percent of identical matches between the smaller of two or more sequences with gap alignments (if any) addressed by a particular mathematical model or computer program (i.e., “algorithms”) Identity of related nucleic acid sequences or polypeptides can be readily calculated by known methods. Such methods include, but are not limited to, those described in Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part 1, Griffin, A. M., and Griffin, H. G., eds., Humana Press, New Jersey, 1994; Sequence Analysis in Molecular Biology, von Heinje, G., Academic Press, 1987; Sequence Analysis Primer, Gribskov, M. and Devereux, J., eds., M. Stockton Press, New York, 1991; and Carillo et al., SIAM J. Applied Math. 48, 1073 (1988). Preferred methods for determining identity are designed to give the largest match between the sequences tested. Methods of determining identity are described in publicly available computer programs. Preferred computer program methods for determining identity between two sequences include ClustalO (Sievers F., et al., 2011), the GCG program package, including, GAP (Devereux et al., Nucl. Acid. Res. \2, 387 (1984); Genetics Computer Group, University of Wisconsin, Madison, Wis.), BLASTP, BLASTN, and FASTA (Altschul et al., J. Mol. Biol. 215, 403-410 (1990)). The BLASTX program is publicly available from the National Center for Biotechnology Information (NCBI) and other sources (BLAST Manual, Altschul et al., NCB/NLM/NIH Bethesda, Md. 20894; Altschul et al., supra). The well-known Smith Waterman algorithm may also be used to determine identity.

In one embodiment, CRM₁₉₇ is full-length CRM₁₉₇.

In one embodiment, the immunogenic product of the invention comprises a fragment of CRM₁₉₇, such as, for example, a fragment comprising at least about 50, 100, 150, 200, 250, 300, 350, 400, 450, or 500 amino acids (preferably contiguous amino acids) from SEQ ID NO: 1.

IgE is an immunoglobulin comprising one variable domain and four constant domains, named Cε1, Cε2, Cε3 and Cε4. IgE further comprises linkers between the different domains.

In one embodiment, the IgE fragment comprises at least one (e.g., 1, 2, 3 or 4) constant domain of IgE. In one embodiment, the IgE fragment does not comprise the variable domain of IgE. In one embodiment, the IgE or fragment thereof does not comprise or consist in full-length IgE.

In one embodiment, the IgE fragment is the full-length IgE constant region (i.e., it comprises the Cε1, Cε2, Cε3 and Cε4 domains and all linker regions).

In one embodiment, the IgE fragment is a fragment of the IgE constant region, such as, for example, a fragment of the IgE constant region comprising at least about 100, 125, 150, 175, 200, 225, 250, 275, 300, 325, 350, 375, 400, or 425 amino acids (preferably contiguous amino acids) of the IgE from which it derives.

In one embodiment, said fragment comprises at least one specific epitope of the IgE constant region.

In one embodiment, the IgE fragment comprises or consists of the Cε3 domain of IgE. In one embodiment, the IgE fragment comprises or consists of at least a part of the Cε3 domain of IgE. In one embodiment, said fragment comprises at least one specific epitope of the IgE Cε3 domain.

In one embodiment, the IgE fragment comprises or consists of the Cε2 and Cε3 domains of IgE. In one embodiment, the IgE fragment comprises or consists of at least a part of the Cε2 and Cε3 domains of IgE.

In one embodiment, the IgE fragment comprises or consists of the Cε2 and Cε3 domains of IgE and the linker region between Cε2 and Cε3. In one embodiment, the IgE fragment comprises or consists of at least a part of the Cε2 and Cε3 domains of IgE and the linker region between Cε2 and Cε3.

In one embodiment, the IgE fragment comprises or consists of the Cε2 and Cε3 domains of IgE and at least one of the linker regions between Cε2 and Cε3, the linker region between Cε1 and Cε2 and the linker region between Cε3 and Cε4. In one embodiment, the IgE fragment comprises or consists of the Cε2 and Cε3 domains of IgE, the linker region between Cε2 and Cε3, the linker region between Cε1 and Cε2 and the linker region between Cε3 and Cε4. In one embodiment, the IgE fragment comprises or consists of at least a part of the Cε2 and Cε3 domain of IgE and at least one of the linker regions between Cε2 and Cε3, the linker region between Cε1 and Cε2 and the linker region between Cε3 and Cε4.

In one embodiment, the IgE fragment comprises or consists of the Cε3 and Cε4 domains of IgE. In one embodiment, the IgE fragment comprises or consists of at least a part of the Cε3 and Cε4 domains of IgE.

In one embodiment, the IgE fragment comprises or consists of the Cε3 and Cε4 domains of IgE and the linker region between Cε3 and Cε4. In one embodiment, the IgE fragment comprises or consists of at least a part of the Cε3 and Cε4 domains of IgE and the linker region between Cε3 and Cε4.

In one embodiment, the IgE fragment comprises or consists of the Cε3 and Cε4 domains of IgE and at least one of the linker regions between Cε2 and Cε3, the linker region between Cε3 and Cε4 and the linker region after Cε4. In one embodiment, the IgE fragment comprises or consists of the Cε3 and Cε4 domains of IgE, the linker region between Cε2 and Cε3, the linker region between Cε3 and Cε4 and the linker region after Cε4. In one embodiment, the IgE fragment comprises or consists of at least a part of the Cε3 and Cε4 domain of IgE at least one of the linker regions between Cε2 and Cε3, the linker region between Cε3 and Cε4 and the linker region after Cε4.

In one embodiment, the IgE fragment comprises or consists of the Cε2, Cε3 and Cε4 domains of IgE. In one embodiment, the IgE fragment comprises or consists of at least a part of the Cε2, Cε3 and Cε4 domains of IgE.

In one embodiment, the IgE fragment comprises or consists of the Cε2, Cε3 and Cε4 domains of IgE and the linker regions between Cε2 and Cε3 and between Cε3 and Cε4. In one embodiment, the IgE fragment comprises or consists of at least a part of the Cε2, Cε3 and Cε4 domain of IgE and the linker regions between Cε2 and Cε3 and between Cε3 and Cε4.

In one embodiment, the IgE fragment comprises or consists of the Cε2, Cε3 and Cε4 domains of IgE, and at least one of the linker regions between Cε1 and Cε2, the linker region between Cε2 and Cε3, the linker region between Cε3 and Cε4 and the linker region after Cε4. In one embodiment, the IgE fragment comprises or consists of the Cε2, Cε3 and Cε4 domains of IgE, the linker region between Cε1 and Cε2, the linker region between Cε2 and Cε3, the linker region between Cε3 and Cε4 and the linker region after Cε4. In one embodiment, the IgE fragment comprises or consists of at least a part of the Cε2, Cε3 and Cε4 domain of IgE and at least one of the linker regions between Cε1 and Cε2, the linker region between Cε2 and Cε3, the linker region between Cε3 and Cε4 and the linker region after Cε4.

In one embodiment, IgE or the IgE fragment is recombinant. Recombinant IgE or fragment thereof may be obtained by conventional methods known in the art using the nucleic sequence encoding IgE or a fragment thereof.

For example, recombinant IgE may be obtained by culturing cells containing an expression vector comprising a nucleic acid sequence (e.g., the gene) encoding IgE, harvesting inclusion bodies and purifying IgE. Accordingly, a recombinant IgE fragment may be obtained by culturing cells containing an expression vector comprising a nucleic acid sequence encoding the IgE fragment, harvesting inclusion bodies and purifying the IgE fragment.

In one embodiment, a recombinant IgE is obtained and a fragment of said recombinant IgE is recovered, for example by proteolysis.

In one embodiment of the present invention, IgE or the fragment thereof is derived from a mammal.

In one embodiment, IgE or the fragment thereof is a variant of a mammal IgE or the fragment thereof, wherein said variant presents at least about 70%, 75, 80, 85, 90, 95% or more identity with the mammal IgE or the fragment thereof from which it derives.

IgE is an immunoglobulin comprising sites of glycosylation. In one embodiment, the IgE or fragment thereof comprised in the immunogenic product of the invention is glycosylated. Without willing to be bound to any theory, the Applicants suggest that the administration of an immunogenic product comprising glycosylated IgE (or a fragment thereof) may induce the production of anti-IgE antibodies specific of the glycosylated form of IgE, corresponding to the native immunoglobulin.

In addition, IgE may be sialylated. Sialylation of hIgE may for example have a role in IgE effector functions. In one embodiment, the IgE or fragment thereof comprised in the immunogenic product of the invention comprises at least one sialic acid residue. In another embodiment, the IgE or fragment thereof comprised in the immunogenic product of the invention do not comprise any sialic acid residue.

In one embodiment, IgE is human IgE, preferably recombinant human IgE. Human IgE constant region has a sequence SEQ ID NO: 2 (UniProt ID: P01854), wherein amino acids from position 6-103, 112-210, 214-318 and 324-423 corresponds respectively to domains Cε1, Cε2, Cε3 and Cε4 Amino acids from position 1-5, 104-111, 211-213, 319-323 and 424-428 correspond respectively to the linker regions before Cε1, between Cε1 and Cε2, Cε2 and Cε3, Cε3 and Cε4 and after Cε4.

SEQ ID NO: 2
ASTQSPSVFPLTRCCKNIPSNATSVTLGCLATGYFPEPVMVTWDTGSLN
GTTMTLPATTLTLSGHYATISLLTVSGAWAKQMFTCRVAHTPSSTDWVD
NKTFSVCSRDFTPPTVKILQSSCDGGGHFPPTIQLLCLVSGYTPGTINI
TWLEDGQVMDVDLSTASTTQEGELASTQSELTLSQKHWLSDRTYTCQVT
YQGHTFEDSTKKCADSNPRGVSAYLSRPSPFDLFIRKSPTITCLVVDLA
PSKGTVNLTWSRASGKPVNHSTRKEEKQRNGTLTVTSTLPVGTRDWIEG
ETYQCRVTHPHLPRALMRSTTKTSGPRAAPEVYAFATPEWPGSRDKRTL
ACLIQNFMPEDISVQWLHNEVQLPDARHSTTQPRKTKGSGFFVFSRLEV
TRAEWEQKDEFICRAVHEAASPSQTVQRAVSVNPGK

In one embodiment, the IgE fragment is a fragment of SEQ ID NO: 2, wherein said fragment comprises at least about 100, 125, 150, 175, 200, 225, 250, 275, 300, 325, 350, 375, 400, or 425 amino acids (preferably contiguous amino acids) of SEQ ID NO: 2.

In one embodiment, the IgE fragment is a variant of SEQ ID NO: 2, wherein said variant presents at least about 70%, 75, 80, 85, 90, 95% or more identity with SEQ ID NO: 2.

In one embodiment, the IgE fragment comprises at least one specific epitope of the human IgE constant domain, preferably of the human IgE Cε3 domain.

In one embodiment, the IgE fragment is full-length human IgE constant region.

In one embodiment, the IgE fragment comprises or consists of the Cε3 domain of human IgE. In one embodiment, the IgE fragment comprises or consists of at least a part of the Cε3 domain of human IgE. In one embodiment, said fragment comprises at least one specific epitope of the human IgE Cε3 domain.

In one embodiment, the IgE fragment comprises or consists of the Cε2 and Cε3 domains of human IgE. In one embodiment, the IgE fragment comprises or consists of at least a part of the Cε2 and Cε3 domains of human IgE.

In one embodiment, the IgE fragment comprises or consists of the Cε2 and Cε3 domains of human IgE and the linker region between Cε2 and Cε3. In one embodiment, the IgE fragment comprises or consists of at least a part of the Cε2 and Cε3 domains of human IgE and the linker region between Cε2 and Cε3.

In one embodiment, the IgE fragment comprises or consists of the Cε2 and Cε3 domains of human IgE and at least one of the linker regions between Cε2 and Cε3, the linker region between Cε1 and Cε2 and the linker region between Cε3 and Cε4. In one embodiment, the IgE fragment comprises or consists of the Cε2 and Cε3 domains of human IgE, the linker region between Cε2 and Cε3, the linker region between Cε1 and Cε2 and the linker region between Cε3 and Cε4. In one embodiment, the IgE fragment comprises or consists of at least a part of the Cε2 and Cε3 domain of human IgE and at least one of the linker regions between Cε2 and Cε3, the linker region between Cε1 and Cε2 and the linker region between Cε3 and Cε4.

In one embodiment, the IgE fragment comprises or consists of the Cε3 and Cε4 domains of human IgE. In one embodiment, the IgE fragment comprises or consists of at least a part of the Cε3 and Cε4 domains of human IgE.

In one embodiment, the IgE fragment comprises or consists of the Cε3 and Cε4 domains of human IgE and the linker region between Cε3 and Cε4. In one embodiment, the IgE fragment comprises or consists of at least a part of the Cε3 and Cε4 domains of human IgE and the linker region between Cε3 and Cε4.

In one embodiment, the IgE fragment comprises or consists of the Cε3 and Cε4 domains of human IgE and at least one of the linker regions between Cε2 and Cε3, the linker region between Cε3 and Cε4 and the linker region after Cε4. In one embodiment, the IgE fragment comprises or consists of the Cε3 and Cε4 domains of human IgE, the linker region between Cε2 and Cε3, the linker region between Cε3 and Cε4 and the linker region after Cε4. In one embodiment, the IgE fragment comprises or consists of at least a part of the Cε3 and Cε4 domains of human IgE at least one of the linker regions between Cε2 and Cε3, the linker region between Cε3 and Cε4 and the linker region after Cε4.

In one embodiment, the IgE fragment comprises or consists of the Cε2, Cε3 and Cε4 domains of human IgE. In one embodiment, the IgE fragment comprises or consists of at least a part of the Cε2, Cε3 and Cε4 domains of human IgE.

In one embodiment, the IgE fragment comprises or consists of the Cε2, Cε3 and Cε4 domains of human IgE and the linker regions between Cε2 and Cε3 and between Cε3 and Cε4. In one embodiment, the IgE fragment comprises or consists of at least a part of the Cε2, Cε3 and Cε4 domain of human IgE and the linker regions between Cε2 and Cε3 and between Cε3 and Cε4.

In one embodiment, the IgE fragment comprises or consists of the Cε2, Cε3 and Cε4 domains of human IgE, and at least one of the linker regions between Cε1 and Cε2, the linker region between Cε2 and Cε3, the linker region between Cε3 and Cε4 and the linker region after Cε4. In one embodiment, the IgE fragment comprises or consists of the Cε2, Cε3 and Cε4 domains of human IgE, the linker region between Cε1 and Cε2, the linker region between Cε2 and Cε3, the linker region between Cε3 and Cε4 and the linker region after Cε4. In one embodiment, the IgE fragment comprises or consists of at least a part of the Cε2, Cε3 and Cε4 domain of human IgE and at least one of the linker regions between Cε1 and Cε2, the linker region between Cε2 and Cε3, the linker region between Cε3 and Cε4 and the linker region after Cε4.

In one embodiment, the IgE fragment comprises or consists in the Cε3 constant domain of human IgE, having the sequence of SEQ ID NO: 3.

SEQ ID NO: 3
PRGVSAYLSRPSPFDLFIRKSPTITCLVVDLAPSKGTVNLTWSRASGKP
VNHSTRKEEKQRNGTLTVTSTLPVGTRDWIEGETYQCRVTHPHLPRALM
RSTTKTS

In one embodiment, the IgE fragment is a fragment of the human IgE Cε3 domain, such as, for example, a fragment of human IgE comprising at least about 50, 55, 60, 65, 70, 75, 80, 85, 90, 95, or 100 amino acids (preferably contiguous amino acids) of SEQ ID NO: 3.

In one embodiment, the IgE fragment is a variant of SEQ ID NO: 3, wherein said variant presents at least about 70%, 75, 80, 85, 90, 95% or more identity with SEQ ID NO: 3.

In one embodiment, the IgE fragment comprises or consists in the Cε3 constant domain of human IgE comprising a mutation at position 3 in SEQ ID NO: 3 (replacement of the Glycine residue with a Cysteine residue). This mutation may be referred to the mutation G335C (Wurzburg et al., 2012). Therefore, in one embodiment, the IgE fragment comprises or consists in SEQ ID NO: 8.

SEQ ID NO: 8
PRCVSAYLSRPSPFDLFIRKSPTITCLVVDLAPSKGTVNLTWSRASGKP
VNHSTRKEEKQRNGTLTVTSTLPVGTRDWIEGETYQCRVTHPHLPRALM
RSTTKTS

In one embodiment, the IgE fragment is a fragment of SEQ ID NO: 8, such as, for example, a fragment of human IgE comprising at least about 50, 55, 60, 65, 70, 75, 80, 85, 90, 95, or 100 amino acids (preferably contiguous amino acids) of SEQ ID NO: 8.

In one embodiment, the IgE fragment is a variant of SEQ ID NO: 8, wherein said variant presents at least about 70%, 75, 80, 85, 90, 95% or more identity with SEQ ID NO: 8.

In one embodiment, the IgE fragment comprises or consists in a fragment of human IgE comprising the Cε3 and Cε4 constant domains, and optionally the linker region between Cε3 and Cε4. An example of a fragment of human IgE comprising the Cε3 and Cε4 constant domains and the linker region between Cε3 and Cε4 has the sequence of SEQ ID NO: 4.

SEQ ID NO: 4
PRGVSAYLSRPSPFDLFIRKSPTITCLVVDLAPSKGTVNLTWSRASGKP
VNHSTRKEEKQRNGTLTVTSTLPVGTRDWIEGETYQCRVTHPHLPRALM
RSTTKTSGPRAAPEVYAFATPEWPGSRDKRTLACLIQNFMPEDISVQWL
HNEVQLPDARHSTTQPRKTKGSGFFVFSRLEVTRAEWEQKDEFICRAVH
EAASPSQTVQRAVS

In one embodiment, the IgE fragment is a fragment of SEQ ID NO: 4, such as, for example, a fragment of human IgE comprising at least about 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200 or 205 amino acids (preferably contiguous amino acids) of SEQ ID NO: 4.

In one embodiment, the IgE fragment is a variant of SEQ ID NO: 4, wherein said variant presents at least about 70%, 75, 80, 85, 90, 95% or more identity with SEQ ID NO: 4.

In one embodiment, the IgE fragment comprises or consists in a fragment of human IgE comprising the Cε3 and Cε4 constant domains and optionally the linker region between Cε3 and Cε4 further comprising a mutation at position 3 in SEQ ID NO: 4 (replacement of the Glycine residue with a Cysteine residue). An example of such an IgE fragment comprising the Cε3 and Cε4 constant domains and the linker region between Cε3 and Cε4 includes an IgE fragment comprising or consisting in SEQ ID NO: 9.

SEQ ID NO: 9
PRCVSAYLSRPSPFDLFIRKSPTITCLVVDLAPSKGTVNLTWSRASGKP
VNHSTRKEEKQRNGTLTVTSTLPVGTRDWIEGETYQCRVTHPHLPRALM
RSTTKTSGPRAAPEVYAFATPEWPGSRDKRTLACLIQNFMPEDISVQWL
HNEVQLPDARHSTTQPRKTKGSGFFVFSRLEVTRAEWEQKDEFICRAVH
EAASPSQTVQRAVS

In one embodiment, the IgE fragment is a fragment of SEQ ID NO: 9, such as, for example, a fragment of human IgE comprising at least about 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200 or 205 amino acids (preferably contiguous amino acids) of SEQ ID NO: 9.

In one embodiment, the IgE fragment is a variant of SEQ ID NO: 9, wherein said variant presents at least about 70%, 75, 80, 85, 90, 95% or more identity with SEQ ID NO: 9.

In one embodiment, the IgE fragment comprises or consists in a fragment of human IgE comprising the Cε2 and Cε3 constant domains and optionally the linker region between Cε2 and Cε3. An example of a fragment of human IgE comprising the Cε2 and Cε3 constant domains and the linker region between Cε2 and Cε3 has the sequence of SEQ ID NO: 5.

SEQ ID NO: 5
PTVKILQSSCDGGGHFPPTIQLLCLVSGYTPGTINITWLEDGQVMDVDL
STASTTQEGELASTQSELTLSQKHWLSDRTYTCQVTYQGHTFEDSTKKC
ADSNPRGVSAYLSRPSPFDLFIRKSPTITCLVVDLAPSKGTVNLTWSRA
SGKPVNHSTRKEEKQRNGTLTVTSTLPVGTRDWIEGETYQCRVTHPHLP
RALMRSTTKTS

In one embodiment, the IgE fragment is a fragment of SEQ ID NO: 5, such as, for example, a fragment of human IgE comprising at least about 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200 or 205 amino acids (preferably contiguous amino acids) of SEQ ID NO: 5.

In one embodiment, the immunogenic product is a variant of SEQ ID NO: 5, wherein said variant presents at least about 70%, 75, 80, 85, 90, 95% or more identity with SEQ ID NO: 5.

In one embodiment, the IgE fragment comprises or consists in a fragment of human IgE comprising the Cε2 and Cε3 constant domains and the linker region between Cε2 and Cε3 further comprising a mutation at position 105 in SEQ ID NO: 5 (replacement of the Glycine residue with a Cysteine residue). An example of such an IgE fragment comprising the Cε2 and Cε3 constant domains and the linker region between Cε2 and Cε3 includes an IgE fragment comprising or consisting in SEQ ID NO: 10.

SEQ ID NO: 10
PTVKILQSSCDGGGHFPPTIQLLCLVSGYTPGTINITWLEDGQVMDVDL
STASTTQEGELASTQSELTLSQKHWLSDRTYTCQVTYQGHTFEDSTKKC
ADSNPRCVSAYLSRPSPFDLFIRKSPTITCLVVDLAPSKGTVNLTWSRA
SGKPVNHSTRKEEKQRNGTLTVTSTLPVGTRDWIEGETYQCRVTHPHLP
RALMRSTTKTS

In one embodiment, the IgE fragment is a fragment of SEQ ID NO: 10, such as, for example, a fragment of human IgE comprising at least about 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200 or 205 amino acids (preferably contiguous amino acids) of SEQ ID NO: 10.

In one embodiment, the IgE fragment is a variant of SEQ ID NO: 10, wherein said variant presents at least about 70%, 75, 80, 85, 90, 95% or more identity with SEQ ID NO: 10.

In one embodiment, the IgE fragment comprises or consists in a fragment of human IgE comprising the Cε2, Cε3 and Cε4 constant domains, and optionally the linker regions between Cε2 and Cε3, and between Cε3 and Cε4. An example of a fragment of human IgE comprising the Cε2, Cε3 and Cε4 constant domains and the linker regions between Cε2 and Cε3, and between Cε3 and Cε4 has the sequence of SEQ ID NO: 6.

SEQ ID NO: 6
PTVKILQSSCDGGGHFPPTIQLLCLVSGYTPGTINITWLEDGQVMDVDL
STASTTQEGELASTQSELTLSQKHWLSDRTYTCQVTYQGHTFEDSTKKC
ADSNPRGVSAYLSRPSPFDLFIRKSPTITCLVVDLAPSKGTVNLTWSRA
SGKPVNHSTRKEEKQRNGTLTVTSTLPVGTRDWIEGETYQCRVTHPHLP
RALMRSTTKTSGPRAAPEVYAFATPEWPGSRDKRTLACLIQNFMPEDIS
VQWLHNEVQLPDARHSTTQPRKTKGSGFFVFSRLEVTRAEWEQKDEFIC
RAVHEAASPSQTVQRAVS

In one embodiment, the IgE fragment is a fragment of SEQ ID NO: 6, such as, for example, a fragment of human IgE comprising at least about 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 270, 275, 280, 285, 290, 295, 300, 305 or 310 amino acids (preferably contiguous amino acids) of SEQ ID NO: 6.

In one embodiment, the IgE fragment is a variant of SEQ ID NO: 6, wherein said variant presents at least about 70%, 75, 80, 85, 90, 95% or more identity with SEQ ID NO: 6.

In one embodiment, the IgE fragment comprises or consists in a fragment of human IgE comprising the Cε2, Cε3 and Cε4 constant domains and the linker regions between Cε2 and Cε3, and between Cε3 and Cε4, further comprising a mutation at position 105 in SEQ ID NO: 6 (replacement of the Glycine residue with a Cysteine residue). An example of such an IgE fragment comprising the Cε2, Cε3 and Cε4 constant domains and the linker region between Cε2 and Cε3 and between Cε3 and Cε4 includes a fragment comprising or consisting in SEQ ID NO: 11.

SEQ ID NO: 11
PTVKILQSSCDGGGHFPPTIQLLCLVSGYTPGTINITWLEDGQVMDVDL
STASTTQEGELASTQSELTLSQKHWLSDRTYTCQVTYQGHTFEDSTKKC
ADSNPRCVSAYLSRPSPFDLFIRKSPTITCLVVDLAPSKGTVNLTWSRA
SGKPVNHSTRKEEKQRNGTLTVTSTLPVGTRDWIEGETYQCRVTHPHLP
RALMRSTTKTSGPRAAPEVYAFATPEWPGSRDKRTLACLIQNFMPEDIS
VQWLHNEVQLPDARHSTTQPRKTKGSGFFVFSRLEVTRAEWEQKDEFIC
RAVHEAASPSQTVQRAVS

In one embodiment, the IgE fragment is a fragment of SEQ ID NO: 11, such as, for example, a fragment of human IgE comprising at least about 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 270, 275, 280, 285, 290, 295, 300, 305 or 310 amino acids (preferably contiguous amino acids) of SEQ ID NO: 11.

In one embodiment, the IgE fragment is a variant of SEQ ID NO: 11, wherein said variant presents at least about 70%, 75, 80, 85, 90, 95% or more identity with SEQ ID NO: 11.

In one embodiment, the IgE fragment comprises or consists of SEQ ID NO: 12.

SEQ ID NO: 12
ADSNPRGVSAYLSRPSPFDLFIRKSPTITCLVVDLAPSKGTVNLTWSRA
SGKPVNHSTRKEEKQRNGTLTVTSTLPVGTRDWIEGETYQCRVTHPHLP
RALMRSTTKTSGPRAAPEVYAFATPEWPGSRDKRTLACLIQNFMPEDIS
VQWLHNEVQLPDARHSTTQPRKTKGSGFFVFSRLEVTRAEWEQKDEFIC
RAVHEAASPSQTVQRAVSVNPGK

In one embodiment, the immunogenic product comprises a variant of SEQ ID NO: 12, wherein said variant presents at least about 70%, 75, 80, 85, 90, 95% or more identity with SEQ ID NO: 12.

In one embodiment, the IgE fragment comprises or consists of SEQ ID NO: 7.

SEQ ID NO: 7
ADSNPRCVSAYLSRPSPFDLFIRKSPTITCLVVDLAPSKGTVNLTWSRA
SGKPVNHSTRKEEKQRNGTLTVTSTLPVGTRDWIEGETYQCRVTHPHLP
RALMRSTTKTSGPRAAPEVYAFATPEWPGSRDKRTLACLIQNFMPEDIS
VQWLHNEVQLPDARHSTTQPRKTKGSGFFVFSRLEVTRAEWEQKDEFIC
RAVHEAASPSQTVQRAVSVNPGK

In one embodiment, the immunogenic product comprises a variant of SEQ ID NO: 7, wherein said variant presents at least about 70%, 75, 80, 85, 90, 95% or more identity with SEQ ID NO: 7.

Human IgE is an immunoglobulin comprising 7 sites of glycosylation including 3 sites of glycosylation in the Cε1 domain (N140, N168 and N218), 1 site of glycosylation in the Cε2 domain (N265) and 3 sites of glycosylation in the Cε3 domain (N371, N383 and N394). In the art, it was suggested that glycosylation on the N394 residue may be important for the folding of the protein, for stable IgE interactions with FcεRI and for the initiation of anaphylaxis.

In one embodiment, the 7 sites of glycosylation correspond respectively in the sequence of the constant region of hIgE of SEQ ID NO: 2 to residues N21, N49, N99 (in the Cε1 domain), N146 (in the Cε2 domain) and N252, N264, N275 (in the Cε3 domain).

In one embodiment, the hIgE or fragment thereof comprised in the immunogenic product of the invention comprises at least one glycosylation. Without willing to be bound to any theory, the Applicants suggest that the administration of such immunogenic product may induce anti-IgE antibodies against native glycosylated hIgE.

In one embodiment, the hIgE or fragment thereof comprised in the immunogenic product of the invention comprises at least one (e.g., 1, 2, 3, 4, 5, 6 or 7) glycosylation.

In one embodiment, the hIgE or fragment thereof comprised in the immunogenic product of the invention comprises the N394 glycosylation. In one embodiment, the hIgE or fragment thereof comprised in the immunogenic product of the invention comprises the N275 glycosylation (when amino acids are numbered as in SEQ ID NO:2).

In addition, sialylation of hIgE may play a role in IgE effector functions.

In one embodiment, the hIgE or fragment thereof comprised in the immunogenic product of the invention comprises at least one sialic acid residue. In one embodiment, the hIgE or fragment thereof comprised in the immunogenic product of the invention does not comprise any sialic acid residue.

In one embodiment of the present invention, IgE is equine IgE, preferably recombinant equine IgE. Equine IgE constant region has a sequence SEQ ID NO: 13, wherein amino acids from position 1-93, 94-200, 201-307 and 308-419 correspond respectively to domains Cε1, Cε2, Cε3 and Cε4.

SEQ ID NO: 13
VSKQAPLIFPLAACCKDTKTTNITLGCLVKGYFPGAWDAGPLNPSTMTF
PAVFDQTSGLYTTISRVVASGKWAKQKFTCGVVHSQETFNKTFNACIVT
FTPPTVKLFHSSCDPGGDSHTTIQLLCLISDYTPGDIDIVWLIEGQKVD
EQFPTQASMKQEGSWPPTHSELNINQGQWASENTYTCQVTYKDMIFNQA
RKCTESDPPGVSVYLSPPSPLDLYVSKTPKITCLVVDLANVQGLSLNWS
RESGEPLQKHTLATSEQFNKTFSVTSTLPVDTTDWIEGETYKCTVSHPD
LPREVVRSIAKAPGKRLSPEVYVFLPPEEDQSSKDKVTLTCLIQNFFPA
DISVQWRRNNVLIQTDQQATTRPQKANGPDPAFFVFSRLEVSRAEWEQK
NKFACKVVHEALSQRTLQKEVSKDPGK

In one embodiment, the IgE fragment is a fragment of SEQ ID NO: 13, wherein said fragment comprises at least about 100, 125, 150, 175, 200, 225, 250, 275, 300, 325, 350, 375, 400, or 415 amino acids (preferably contiguous amino acids) of SEQ ID NO: 13.

In one embodiment, the IgE fragment is a variant of SEQ ID NO: 13, wherein said variant presents at least about 70%, 75, 80, 85, 90, 95% or more identity with SEQ ID NO: 13.

In one embodiment of the present invention, IgE is canine IgE, preferably recombinant canine IgE. Canine IgE constant region has a sequence SEQ ID NO: 14.

SEQ ID NO: 14
TSQDLSVFPLASCCKDNIASTSVTLGCLVTGYLPMSTTVTWDTGSLNKN
VTTFPTTFHETYGLHSIVSQVTASGKWAKQRFTCSVAHAESTAINKTFS
ACALNFIPPTVKLFHSSCNPVGDTHTTIQLLCLISGYVPGDMEVIWLVD
GQKATNIFPYTAPGTKEGNVTSTHSELNITQGEWVSQKTYTCQVTYQGF
TFKDEARKCSESDPRGVTSYLSPPSPLDLYVHKAPKITCLVVDLATMEG
MNLTWYRESKEPVNPGPLNKKDHFNGTITVTSTLPVNTNDWIEGETYYC
RVTHPHLPKDIVRSIAKAPGKRAPPDVYLFLPPEEEQGTKDRVTLTCLI
QNFFPADISVQWLRNDSPIQTDQYTTTGPHKVSGSRPAFFIFSRLEVSR
VDWEQKNKFTCQVVHEALSGSRILQKWVSKTPGK

In one embodiment, the IgE fragment is a fragment of SEQ ID NO: 14, wherein said fragment comprises at least about 100, 125, 150, 175, 200, 225, 250, 275, 300, 325, 350, 375, 400, or 425 amino acids (preferably contiguous amino acids) of SEQ ID NO: 14.

In one embodiment, the IgE fragment is a variant of SEQ ID NO: 14, wherein said variant presents at least about 70%, 75, 80, 85, 90, 95% or more identity with SEQ ID NO: 14.

In one embodiment of the present invention, IgE is feline IgE, preferably recombinant feline IgE. Feline IgE constant region has a sequence SEQ ID NO: 15.

SEQ ID NO: 15
AYISSGGNTDYADSVKGRFSISRDNAKNTLYLQMTSLKTEDTATYYCAR
GTGVIPDYWGQGALVTVSSTSIQAPLVFPLATCCKGTIATAPSVTLGCL
VTGYFPMPVTVTWDARSLNKSVVTLPATLQENSGLYTTTSHVTVSGEWA
KQKFTCSVAHAESPTINKTVSACTMNFIPPTVKLFHSSCNPLGDTGSTI
QLLCLISGYVPGDMEVTWLVDGQKATNIFPYTAPGKQEGKVTSTHSELN
ITQGEWVSQKTYTCQVTYQGFTFEDHARKCTESDPRGVSTYLSPPSPLD
LYVHKSPKITCLVVDLANTDGMILTWSRENGESVHPDPMVKKTQYNGTI
TVTSTLPVDATDWVEGETYQCKVTHPDLPKDIVRSIAKAPGRRFPPEVY
VFLPPEGEPKTKDKVTLTCLIQNFFPPDISVQWLHNDSPVRTEQQATTW
PHKATGPSPAFFVFSRLEVSRADWEQRDVFTCQVVHEALPGFRTLKKSV
SKNPGK

In one embodiment, the IgE fragment is a fragment of SEQ ID NO: 15, wherein said fragment comprises at least about 100, 125, 150, 175, 200, 225, 250, 275, 300, 325, 350, 375, 400, 425, 450, 475 or 490 amino acids (preferably contiguous amino acids) of SEQ ID NO: 15.

In one embodiment, the IgE fragment is a variant of SEQ ID NO: 15, wherein said variant presents at least about 70%, 75, 80, 85, 90, 95% or more identity with SEQ ID NO: 15.

In one embodiment, the immunogenic product of the invention comprises IgE or a fragment thereof coupled to a carrier protein, preferably to CRM₁₉₇, at a molar ratio IgE (or fragment thereof):CRM₁₉₇ ranging from about 16:1 to about 1:4, preferably from about 8:1 to about 1:2, more preferably of about 1:1.

In one embodiment, the immunogenic product of the invention comprises IgE or an IgE fragment coupled to a carrier protein, preferably to CRM₁₉₇, and is recognized by anti-IgE antibodies.

The fact that the immunogenic product comprises IgE or an IgE fragment coupled to a carrier protein, preferably CRM₁₉₇, and is recognized by anti-IgE antibodies may be verified by conventional methods known in the art. An example of such methods is a sandwich ELISA anti-IgE or fragment thereof/carrier protein, using for example a detection antibody labelled with biotin, streptavidin HRP amplification system and an o-phenylenediamine dihydrochloride (OPD) substrate solution.

In one embodiment, the immunogenic product of the invention comprises IgE or a fragment thereof coupled to a carrier protein, preferably to CRM₁₉₇, and is immunogenic, which means that the immunogenic product is capable of inducing anti-IgE antibodies in vivo in the conditions of TEST A. In one embodiment, the immunogenic product of the invention is capable of inducing polyclonal anti-IgE antibodies in vivo, such as, for example, in the conditions of TEST A.

TEST A is carried out according to the following method:

Specific amounts of total proteins (as determined, for example, by a Bradford protein assay) of the immunogenic product are injected in mice (older than 3 weeks of age), three times in 28 days. In one embodiment, TEST A comprises administering a dose of total proteins ranging from about 10 to 30 μg. Serum samples are obtained before immunization (pre-immune serum sample) and after immunization (test serum sample). ELISA anti-IgE are carried out as explained below.

Briefly, human IgE or CRM₁₉₇ are coated at 4° C. at 5 or 1 μg/mL respectively in coating buffer (carbonate/bicarbonate buffer pH 9.6) and incubated overnight. After each step, plates are washed three times with PBS Tween 20 at 0.005%. After blocking with BSA 1% PBS, serum samples are added, a two-fold serial dilution was conducted starting at 2000 dil⁻¹ (diluted in PBS, BSA 1%). After 90 minutes of incubation at 37° C., bound antibodies are detected with HRP-conjugated goat anti mouse IgG (Bethyl Laboratories) at 1/10 000 and plates are revealed using an OPD substrate. Reaction is stopped with 1 M H₂SO₄ and absorbance is subsequently recorded at 490 nm. Samples are analyzed starting at dilution 2000 dil⁻¹ up to 1 024 000 dil⁻¹.

In one embodiment, when the optical density of wells (490 nm) containing the test serum sample is at least about 2-fold, preferably at least about 5-fold superior to the optical density of wells containing the pre-immune serum sample, the immunogenic product is considered as immunogenic, which means that it has induced anti-IgE antibodies in vivo.

In this test, the titers may be defined as the dilution of the serum where 50% of the ODmax minus OD of corresponding preimmune sample in the assay is reached. This mode of calculation may be much more stringent than looking at the well-known seroconversion titers but may provide more robust analysis and less false positive. Titers may be expressed as serum dilution factors (dil⁻¹).

In one embodiment, in TEST A, a titer value ≥1000 dil⁻¹, preferably ≥2000 dil⁻¹ indicates that the immunogenic product of the invention allows the production of binding antibodies against IgE.

In one embodiment, the immunogenic product of the invention comprises IgE or a fragment thereof coupled to CRM₁₉₇ and is capable of neutralizing IgE activity in condition of hereunder cited TEST B. According to the invention, TEST B is performed to evaluate the neutralizing capacity of the serum obtained from mice immunized with the immunogenic product.

TEST B is carried out according to the following method:

Bone marrow-derived cultured mast cells (BMCMCs) expressing hFcεRI are obtained by culturing bone marrow cells from IgE/FcεRI humanized mice in medium containing IL-3 (10 ng/ml) for 6 weeks, at which time cells were >95% c-Kit⁺hFcεRIα⁺. To assess the neutralizing capacity of anti-hIgE antibodies produced upon vaccination with an immunogenic product of the invention, BMCMCs are incubated with dilutions of plasma from mice vaccinated with the immunogenic product of the invention or with CRM₁₉₇ alone. Then FITC-labeled hIgE is added, and the binding of FITC-hIgE to hFcεRI on BCMMCs is assessed by flow cytometry.

Results may be expressed as percentage of IgE-FITC-positive BMCMCs (used as a readout of hIgE binding). A NC₅₀ can be determined in this test by interpolating the plasma dilution resulting in a 50% of IgE-FITC binding on the abscissa axis.

In one embodiment, in TEST B, a NC₅₀ value ≥100 dil⁻¹, preferably ≥200 dil⁻¹ indicates that the immunogenic product of the invention allows the production of neutralizing antibodies against IgE. In one embodiment, the neutralizing antibodies against IgE induced by the administration of the immunogenic product of the invention are polyclonal.

The present invention further relates to a method for producing an immunogenic product comprising at least one IgE or fragment thereof coupled with a carrier protein, preferably CRM₁₉₇, wherein preferably the IgE fragment comprises the IgE Cε3 domain, wherein the method comprises the following steps:
a) contacting the at least one IgE or fragment thereof with a heterobifunctional crosslinker containing a NHS-ester, preferably N-[γ-maleimidobutyryloxy]-succinimide ester (sGMBS), thereby obtaining a complex between a heterobifunctional crosslinker containing a NHS-ester and the immunoglobulin or fragment thereof, preferably a sGMBS-immunoglobulin or fragment thereof complex;
b) contacting the carrier protein with a heterobifunctional crosslinker containing a NHS-ester, preferably N-succinimidyl-S-acetylthioacetate (SATA) to generate a complex between the heterobifunctional crosslinker containing a NHS-ester and the carrier, preferably a carrier-SATA complex;
c) contacting the complex between a heterobifunctional crosslinker containing a NHS-ester and the immunoglobulin or fragment thereof, preferably the sGMBS-IgE complex obtained at step (a) with the complex between the heterobifunctional crosslinker containing a NHS-ester and the carrier, preferably the carrier-SATA complex obtained at step (b).

In one embodiment, in step a), the reaction buffer is in a liquid, preferably aqueous, solution.

In one embodiment, in step a), the reaction buffer is at a pH ranging from about 6 to about 8, preferably ranging from about 6.5 to about 7.5, more preferably at about pH 7.2.

In one embodiment, in step a), the IgE or fragment thereof is present in solution at a concentration ranging from about 0.1 to about 10 mg/mL, preferably from about 0.5 to about 5 mg/ml, more preferably of about 1 mg/mL.

In one embodiment, in step a), the heterobifunctional crosslinker containing a NHS-ester, preferably sGMBS, is prepared in reaction buffer at a concentration ranging from 1 mM to 100 mM, preferably from 5 mM to 50 mM and more preferably at 10 mM.

In one embodiment, in step a), IgE or the fragment thereof and the heterobifunctional crosslinker containing a NHS-ester, preferably sGMBS, are mixed at a IgE (or fragment thereof):heterobifunctional crosslinker containing a NHS-ester, preferably sGMBS, molar ratio ranging from about 1:120 to about 1:1, preferably from about 1:50 to about 1:10, more preferably from about 1:40 to about 1:20.

In one embodiment, in step a), the at least one IgE or fragment thereof is incubated with the heterobifunctional crosslinker containing a NHS-ester, preferably sGMBS, for a period ranging from about 30 min to about 120 min, preferably from about 45 to about 90 minutes and more preferably during at least 60 minutes.

In one embodiment, in step a), the contacting step of the at least one IgE or fragment thereof with the heterobifunctional crosslinker containing a NHS-ester, preferably sGMBS, is performed at a temperature ranging from about 15° C. to about 35° C., preferably from about 18° C. to about 27° C.

In one embodiment, following step a), small compounds having a molecular weight of less than about 10 kDa, less than about 5 kDa or less than about 3 kDa that are present in the reaction mixture are removed. These small compounds encompass mainly the excess of the heterobifunctional crosslinker containing a NHS-ester (and NHS-ester hydrolysis-related side-products), preferably sGMBS, and the excess molecules that have not reacted. Such removing may be performed by methods well known in the art (see the Example part for an example of such method).

In one embodiment, at the end of step a), the protein content is determined by Bradford assay or by any method well known in the art.

In one embodiment, in step b), the reaction buffer is in a liquid, preferably aqueous, solution.

In one embodiment, in step b), the reaction buffer is at a pH ranging from about 6 to about 8, preferably ranging from about 6.5 to about 7.5, more preferably at about pH 7.2.

In one embodiment, in step b), the carrier protein, preferably CRM₁₉₇, is present in solution at a concentration ranging from about 0.2 to about 20 mg/mL, preferably from about 1 to about 10 mg/ml, more preferably of about 2 mg/mL.

In one embodiment, in step b), the heterobifunctional crosslinker containing a NHS-ester, preferably SATA, is present in solution, preferably in DMSO, at a concentration ranging from 20 mM to about 500 mM, preferably from about 50 mM to about 200 mM and more preferably at a concentration of about 100 mM.

In on embodiment, in step b), the carrier protein, preferably CRM₁₉₇, and the heterobifunctional crosslinker containing a NHS-ester, preferably SATA, are mixed at a carrier: heterobifunctional crosslinker containing a NHS-ester, preferably SATA, molar ratio ranging from about 1:320 to about 1:10, preferably from about 1:160 to about 1:40.

In one embodiment, in step b), the carrier protein, preferably CRM₁₉₇, is incubated with the heterobifunctional crosslinker containing a NHS-ester, preferably SATA, for a period of time ranging from about 10 min to about 60 min, preferably from about 15 minutes to about 45 minutes and more preferably during 30 minutes.

In one embodiment, the contacting step b) is performed at a temperature ranging from about 15° C. to about 35° C., preferably from about 18° C. to about 27° C.

In one embodiment, following step b), small compounds having a molecular weight of less than about 10 kDa, less than about 5 kDa or less than about 3 kDa that are present in the reaction mixture are removed. These small compounds encompass mainly the excess of the heterobifunctional crosslinker containing a NHS-ester (and NHS-ester hydrolysis-related side-products), preferably SATA, DMSO, and the excess molecules that have not reacted. Such removing may be performed by methods well known in the art.

In one embodiment, after step b), the complexes between the carrier protein, preferably CRM₁₉₇, and the heterobifunctional crosslinker containing a NHS-ester, preferably SATA, are deprotected to convert the protecting group (the heterobifunctional crosslinker containing a NHS-ester, preferably SATA) into a functional group. In one embodiment, said deprotecting step is carried out after a step of removing small compounds having a molecular weight of less than about 10 kDa, less than about 5 kDa or less than about 3 kDa that are present in the reaction mixture.

Examples of methods for deprotecting a molecule are well known in the art and include, without limitation, the use of hydroxylamine, the use of methoxylamine, or the use of a base (such as, for example, NaOH, KOH, K₂CO₃, MeONa, NH₃ in methanol).

In one embodiment, the deprotecting step comprises the addition to the reaction mixture of a hydroxylamine solution, preferably at a final concentration ranging from about 10 mM to about 500 mM, preferably from about 20 mM to about 100 mM, more preferably at about 50 mM.

In one embodiment, the hydroxylamine solution is incubated with the reaction mixture for a period of time ranging from about 60 min to about 180 min, preferably from about 90 minutes to about 150 minutes, and more preferably during 120 minutes.

In one embodiment, the hydroxylamine solution is added at 50 mM during 120 minutes.

In one embodiment, the incubation of the hydroxylamine solution with the reaction mixture is performed at a temperature ranging from about 15° C. to about 35° C., preferably from about 18° C. to about 27° C.

In one embodiment, following the deprotection step, small compounds having a molecular weight of less than about 10 kDa, 5 kDa or 3 kDa that are present in the reaction mixture are removed. These small compounds encompass mainly the excess of hydroxylamine and potential residual SATA from the previous step. Such removing may be performed by methods well known in the art.

In one embodiment, at the end of step b), the protein content is determined by Bradford assay or by any method well known in the art.

Then, in step c) of the method of the invention, the final product of step a) is contacted with the final product of step b), thereby producing the immunogenic product of the invention.

In one embodiment, in step c), the final product of step a) comprising IgE or an IgE fragment and the final product of step b) comprising the carrier protein, preferably CRM₁₉₇, are contacted at a molar ratio IgE or fragment thereof:carrier protein, preferably CRM₁₉₇ ranging from about 8:1 to about 1:8, preferably from about 4:1 to about 1:4, more preferably of about 1:1.

In one embodiment, in step c), the final product of step a) comprising IgE or an IgE fragment and the final product of step b) comprising the carrier protein, preferably CRM₁₉₇, are contacted at a final protein concentration ranging from about 0.01 to about 5 mg/mL, preferably from about 0.1 to about 1 mg/mL, more preferably of about 0.4 mg/mL.

In one embodiment, in step c), the reaction buffer is in a liquid, preferably aqueous, solution.

In one embodiment, in step c), the reaction buffer is at a pH ranging from about 6 to about 8, preferably ranging from about 6.5 to about 7.5, more preferably at about pH 7.2.

In one embodiment of step c), the contacting step is carried out for a period of time ranging from about 2 hours to about 26 hours, preferably from about 10 to 18 hours, more preferably from about 12 to about 18 hours.

In one embodiment, the incubation step c) is carried out at a temperature ranging from about 2° C. to 10° C., preferably from about 3° C. to about 7° C., and more preferably at about 4° C.

In one embodiment, following step c), small compounds having a molecular weight of less than about 100 kDa, less than about 50 kDa, less than about 25 kDa, less than about 10 kDa, less than about 5 kDa or less than about 3 kDa that are present in the reaction mixture are removed. These small compounds encompass mainly the excess molecules that have not reacted. Such removing may be performed by methods well known in the art.

In one embodiment, the immunogenic product obtained at step c) is concentrated. The concentration of the immunogenic product may be performed by the skilled artisan by any technique known in the art, such as, for example, by a centrifugal ultrafiltration method that may optionally be combined with sterile filtration.

In one embodiment, the immunogenic product obtained at step c) and optionally concentrated is lyophilized.

The present invention further relates to an immunogenic product susceptible to be obtained by the method of the present invention.

The present invention further relates to a composition comprising, consisting essentially of or consisting of at least one immunogenic product as described hereinabove. In one embodiment, said composition may be referred to as an immunogenic composition.

The present invention further relates to a pharmaceutical composition comprising, consisting essentially of or consisting of at least one immunogenic product as described hereinabove, and at least one pharmaceutically acceptable excipient.

Pharmaceutically acceptable excipients that may be used in the pharmaceutical composition of the invention include, but are not limited to, ion exchangers, alumina, aluminum stearate, lecithin, serum proteins, such as, for example, human serum albumin, buffer substances such as, for example, phosphates, glycine, sorbic acid, potassium sorbate, partial glyceride mixtures of saturated vegetable fatty acids, water, salts or electrolytes, such as, for example, protamine sulfate, disodium hydrogen phosphate, potassium hydrogen phosphate, sodium chloride, zinc salts, colloidal silica, magnesium trisilicate, polyvinyl pyrrolidone, cellulose-based substances (for example sodium carboxymethylcellulose), polyethylene glycol, polyacrylates, waxes, polyethylene-polyoxypropylene-block polymers, polyethylene glycol and wool fat.

The present invention further relates to a medicament comprising, consisting essentially of or consisting of at least one immunogenic product as described hereinabove.

As used herein, the term “consisting essentially of”, with reference to a composition, pharmaceutical composition or medicament, means that the at least one immunogenic product of the invention is the only one therapeutic agent or agent with a biologic activity within said composition, pharmaceutical composition or medicament.

In one embodiment, the composition, pharmaceutical composition or medicament of the invention comprises or consists essentially of an immunogenic product comprising IgE or an IgE fragment coupled with a carrier protein, preferably CRM₁₉₇.

In one embodiment, the composition, pharmaceutical composition or medicament of the invention is a vaccine composition. In one embodiment of the invention, the vaccine composition of the invention comprises at least one adjuvant.

This invention further relates to a formulation of the composition, pharmaceutical composition, medicament or vaccine of the invention, wherein the composition, pharmaceutical composition, medicament or vaccine is adjuvanted.

In one embodiment, the composition, pharmaceutical composition, medicament or vaccine of the invention thus comprise one or more adjuvants.

Suitable adjuvants that may be used in the present invention include, but are not limited to:
(1) aluminum salts (alum), such as, for example, aluminum hydroxide, aluminum phosphate, aluminum sulfate, etc.;
(2) oil-in-water emulsion formulations (with or without other specific immunostimulating agents such as, for example, muramyl peptides (defined below) or bacterial cell wall components), such as, for example, squalene-based emulsions (e.g., squalene-based oil-in-water emulsions) or squalane-based emulsions, such as, for example,
(a) MF59 (a squalene-based oil-in-water adjuvant described in PCT Publ. No. WO 90/14837), containing 5% squalene, 0.5% Tween 80, and 0.5% span 85 (optionally containing various amounts of MTP-PE (see below, although not required)) formulated into submicron particles using a microfluidizer such as Model 110Y microfluidizer (Microfluidics, Newton, Mass.),
(b) SWE01 (an oil-in-water squalene-based adjuvant),
(c) SAF, containing 10% Squalene, 0.4% Tween 80, 5% pluronic-blocked polymer L121, and thr-MDP (see below) either microfluidized into a submicron emulsion or vortexed to generate a larger particle size emulsion,
(d) Ribi™ adjuvant system (RAS), (Corixa, Hamilton, Mont.) containing 2% squalene, 0.2% Tween 80, and one or more bacterial cell wall components from the group consisting of 3-O-deacylated monophosphorylipid A (MPL™) described in U.S. Pat. No. 4,912,094 (Corixa), trehalose dimycolate (TDM), and cell wall skeleton (CWS), preferably MPL+CWS (Detox™);
(e) squalane based adjuvant comprising but not limited to the following composition: squalane 3.9%, w/v, sorbitan trioleate (0.47%, w/v), and polyoxyethylene (80) sorbitan monooleate (0.47%, w/v) dispersed in citrate buffer;
(3) water-in-oil emulsion formulations, such as, for example, ISA-51 or squalene-based water-in-oil adjuvant (e.g., ISA-720); Oil adjuvants suitable for use in water-in-oil emulsions may include mineral oils and/or metabolizable oils. Mineral oils may be selected from Bayol®, Marcol® and Drakeol, including Drakeol® 6VR (SEPPIC, France)® Metabolizable oils may be selected from SP oil (hereinafter described), Emulsigen (MPV Laboratories, Ralston, NZ), Montanide 264,266,26 (Seppic SA, Paris, France), as well as vegetable oils, animal oils such as the fish oils squalane and squalene, and tocopherol and its derivatives;
(4) saponin adjuvants, such as Quil A or STIMULON™ QS-21 (Antigenics, Framingham, Mass.) (U.S. Pat. No. 5,057,540) may be used or particles generated therefrom such as ISCOMs (immunostimulating complexes);
(5) bacterial lipopolysaccharides, synthetic lipidA analogs such as aminoalkyl glucosamine phosphate compounds (AGP), or derivatives or analogs thereof, which are available from Corixa, and which are described in U.S. Pat. No. 6,113,918; one such AGP is 2-[(R)-3-Tetradecanoyloxytetradecanoylamino]ethyl 2-Deoxy-4-O-phosphono-3-Oi[(R)-3tetradecanoyloxytetradecanoyl]-2-[(R)-3-tetradecanoyloxytetradecanoyl amino]-b-Dglucopyranoside, which is also known as 529 (formerly known as RC529), which is formulated as an aqueous form or as a stable emulsion, synthetic polynucleotides such as oligonucleotides containing CpG motif(s) (U.S. Pat. No. 6,207,646);
(6) cytokines, such as interleukins (e.g., IL-1, IL-2, IL-4, IL-5, IL-6, IL-7, IL-12, IL-15, IL-18, etc.), interferons (e.g., gamma interferon), granulocyte macrophage colony stimulating factor (GM-CSF), macrophage colony stimulating factor (M-CSF), tumor necrosis factor (TNF), costimulatory molecules B7-1 and B7-2, etc.;
(7) detoxified mutants of a bacterial ADP-ribosylating toxin such as a cholera toxin (CT) either in a wild-type or mutant form, for example, where the glutamic acid at amino acid position 29 is replaced by another amino acid, preferably a histidine, in accordance with published international patent application number WO 00/18434 (see also WO 02/098368 and WO 02/098369), a pertussis toxin (PT), or an E. coli heat-labile toxin (LT), particularly LT-K63, LT-R72, CT-S109, PT-K9/G129 (see, e.g., WO 93/13302 and WO92/19265); and
(8) other substances that act as immunostimulating agents to enhance the effectiveness of the composition. Muramyl peptides include, but are not limited to, N-acetylmuramyl-L-threonyl-D-isoglutamine (thr-MDP), N-acetylnormuramyl-L-alanine-2-(1′-2′dipalmitoyl-sn-glycero-3hydroxyphosphoryloxy)-ethylamine (MTP-PE), etc.

The adjuvant used may depend, in part, on the recipient organism. Moreover, the amount of adjuvant to administer will depend on the type and size of animal.

In one embodiment, the composition, pharmaceutical composition, medicament or vaccine composition of the invention is (or comprises) an emulsion further comprising one or more surfactant agents, and optionally at least one adjuvant as described hereinabove. In one embodiment, the emulsion is a water-in-oil emulsion or an oil-in-water emulsion.

Examples of surfactants that may be used in the present invention are well known in the art and include, but are not limited to, mannide monoleate such as Montanide® 80 marketed by Arlacel (SEPPIC, France), Tween 20, Tween 80, span 85, Triton X-100.

In one embodiment, the composition, pharmaceutical composition, medicament, vaccine composition of the invention comprises a therapeutically effective amount of at least one immunogenic product of the invention.

In one embodiment and for storage purposes, the immunogenic product or the composition, pharmaceutical composition, medicament, or vaccine composition of the invention is lyophilized.

In one embodiment, the composition, pharmaceutical composition, medicament, or vaccine composition of the invention may thus be presented in a freeze-dried (lyophilized) form. According to this embodiment, the immunogenic product of the invention is combined with one or more lyophilization auxiliary substances. Various lyophilization auxiliary substances are well known by the one skilled in the art and include, without limitation, sugars like lactose and mannitol.

In one embodiment, the composition, pharmaceutical composition, medicament, or vaccine composition of the invention may be mixed with stabilizers, e.g., to protect degradation-prone proteins from being degraded, to enhance the shelf-life of the immunogenic product, or to improve freeze-drying efficiency. Useful stabilizers include, but are not limited to, SPGA, carbohydrates (e.g., sorbitol, mannitol, trehalose, starch, sucrose, dextran or glucose), proteins (such as, for example, albumin or casein or degradation products thereof), mixtures of amino acids such as, for example, lysine or glycine, and buffers, such as, for example, alkali metal phosphates.

In one embodiment, the immunogenic product, composition, pharmaceutical composition, or vaccine composition of the invention may be administered by injection, topically (such as, for example, by transdermal delivery), rectally, nasally or vaginally.

In one embodiment, the immunogenic product, composition, pharmaceutical composition, medicament, or vaccine composition of the invention is in an adapted form for an injection. Thus, in one embodiment, the immunogenic product, composition, pharmaceutical composition, medicament, or vaccine composition of the invention is to be injected (or is for injection) to the subject by intramuscular, intraperitoneal, or subcutaneous injection.

Examples of forms suitable for injectable use include, but are not limited to, sterile solutions or dispersions and sterile powders for the extemporaneous preparation of sterile injectable solutions or dispersions. The prevention against contamination by microorganisms can be brought about by adding in the composition preservatives such as, for example, various antibacterial and antifungal agents (for example, parabens, chlorobutanol, phenol, sorbic acid, thimerosal and the like). In an embodiment, it may be preferable to include isotonic agents, for example, sugars or sodium chloride, to reduce pain during injection. In one embodiment, prolonged absorption of the injectable compositions can be brought about by the use in the compositions of agents delaying absorption, for example, aluminum monostearate and gelatin.

In one embodiment, a lyophilized vaccine composition of the invention is solubilized in water for injection and gently mixed; then an immunoadjuvant as described hereinabove, is added; the mixture is gently mixed and charged into a suitable syringe. This invention thus also relates to a medical device, including a syringe filled or prefilled with a vaccine composition of the invention.

In one embodiment, the immunogenic product, composition, pharmaceutical composition, medicament, or vaccine composition of the invention is in an adapted form for topical administration. Examples of forms adapted for topical administration include, without being limited to, polymeric patch, or controlled-release patch, and the like.

In another embodiment, the immunogenic product, composition, pharmaceutical composition, medicament, or vaccine composition of the invention is in an adapted form for rectal administration. Examples of forms adapted for rectal administration include, without being limited to, suppository, micro enemas, enemas, gel, rectal foam, cream, ointment, and the like.

This invention also relates to the medical device which is the syringe filled or prefilled with the composition, pharmaceutical composition, medicament, or vaccine composition of the invention.

In one embodiment, said syringe is a dual chamber syringe, wherein one chamber comprises a solution with the immunogenic product of the invention and the other chamber comprises the adjuvant.

The invention also relates to a medical device comprising a vial prefilled with the immunogenic product of the invention or with the composition, pharmaceutical composition, medicament, or vaccine composition of the invention.

The present invention further relates to the immunogenic product of the invention, for use as a medicament or as a drug.

The present invention further relates to the immunogenic product, composition, pharmaceutical composition, medicament, or vaccine composition of the invention, for treating an inflammatory disorder in a subject.

The present invention further relates to the use of the immunogenic product of the invention for the manufacture of a medicament for treating an inflammatory disorder in a subject.

The present invention thus further relates to a method for treating an inflammatory disorder in a subject, comprising administering to the subject the immunogenic product, composition, pharmaceutical composition, medicament, or vaccine composition of the invention.

The present invention further relates to a method for inducing an immune response against IgE in a subject, comprising administering to the subject the immunogenic product, composition, pharmaceutical composition, medicament, or vaccine composition of the invention.

The present invention further relates to a method for inducing in a subject the production of antibodies that inhibits the biological activity or neutralizes the biological activity of IgE, comprising administering to the subject the immunogenic product, composition, pharmaceutical composition, medicament, or vaccine composition of the invention. In one embodiment, the antibodies are polyclonal antibodies.

In one embodiment, the subject is affected, preferably is diagnosed, with an inflammatory disorder, in particular with an inflammatory disorder associated with aberrant total IgE expression or activity and/or expression of allergen-specific IgE.

In one embodiment, the subject is a human Preferably, according to this embodiment, the at least one IgE or fragment thereof comprised in the immunogenic product of the invention is human.

In one embodiment, the subject is a non-human mammal (such as, for example, a pet). Preferably, according to this embodiment, the at least one IgE or fragment thereof comprised in the immunogenic product of the invention originates from said non-human mammal.

In one embodiment, the subject is a horse, a dog or a cat. Preferably, according to this embodiment, the at least one IgE or fragment thereof comprised in the immunogenic product of the invention is respectively equine, canine or feline.

In one embodiment, the inflammatory disorder is a disorder associated with aberrant IgE expression or activity.

Examples of inflammatory disorder include, but are not limited to, asthma, allergic conditions (such as, for example, food allergies, venom allergy, cat allergy, drug allergy, hyper IgE syndrome, allergic rhinitis, allergic conjunctivitis and allergic enterogastritis), anaphylaxis, atopic disorders (such as, for example, urticaria (including chronic idiopathic urticaria and chronic spontaneous urticaria), eczema), bullous pemphigoid, respiratory disorders (such as asthma, allergic bronchopulmonary aspergillosis, allergic bronchopulmonary mycosis), nasal polyposis and other conditions involving airway inflammation (such as, for example, eosinophilia, fibrosis and excess mucus production including cystic fibrosis and pulmonary fibrosis, systemic sclerosis (SSc)); inflammatory and/or autoimmune disorders or conditions, gastrointestinal disorders or conditions (such as, for example, inflammatory bowel diseases (IBD), and eosinophilic-mediated gastrointestinal disease, ulcerative colitis, Crohn's disease and systemic lupus erythematosus); systemic lupus erythematosus; mastocytosis and mast cell activation syndrome (MCAS).

In one embodiment, the inflammatory disorder is selected from the group comprising asthma (e.g., allergic asthma), allergic rhinitis, allergic conjunctivitis, allergy (e.g., food or venom allergy), anaphylaxis, and nasal polyposis.

In one embodiment, the inflammatory disorder is selected from the group comprising asthma (e.g., allergic asthma), allergy (e.g., food or venom allergy) and anaphylaxis.

In one embodiment, the inflammatory disorder is selected from allergy, anaphylaxis, allergic asthma, allergic rhinitis, allergic conjunctivitis, nasal polyposis, preferably said inflammatory disorder is food or venom allergy.

In one embodiment, the inflammatory disorder is allergic asthma.

The present invention further relates to a method for inducing desensitization of a subject allergic to a specific antigen, wherein said method comprises administering to the subject the immunogenic product, composition, pharmaceutical composition, medicament, or vaccine composition of the invention and said allergen.

As used herein, the term “desensitization”, also known as allergen immunotherapy, desensitization or hypo-sensitization or allergy vaccination, refers to a medical treatment for environmental allergies, such as allergic asthma. Such treatment involves exposing people to larger and larger amounts of allergen in an attempt to reduce the immune system's response in presence of allergen.

Examples of allergens include, but are not limited to inhaled allergens, ingested allergens and contact allergens.

Examples of inhaled allergens include, but are not limited to, allergens from astigmata (e.g., Acarus siro (Storage mite, Aca s 13), Blomia tropicalis (Mite, Blo t), Dermatophagoides farinae (American house dust mite, Der f), Dermatophagoides microceras (House dust mite, Der m), Dermatophagoides pteronyssinus (European house dust mite, Der p), Euroglyphus maynei (House dust mite, Eur m), Glycyphagus domesticus (Storage mite, Gly d 2), Lepidoglyphus destructor (Storage mite, Lep d), Tyrophagus putrescentiae (Storage mite, Tyr p)); Blattaria (e.g., Blattella germanica (German cockroach, Bla g), Periplaneta americana (American cockroach, Per a)); coleoptera (e.g., Harmonia axyridis (Asian ladybeetle, Har a)), Diptera (e.g., Aedes aegypti (Yellow fever mosquito, Aed a), Chironomus kiiensis (Midge, Chi k), Chironomus thummi thummi (Midge, Chi t), Forcipomyia taiwana (Biting midge, For t), Glossina morsitans (Savannah Tsetse fly, Glo m), Hemidiptera: Triatoma protracta (California kissing bug, Tria p)), Hymenoptera (e.g., Apis cerana (Eastern hive bee, Api c), Apis dorsata (Giant honeybee, Api d), Apis mellifera (Honey bee, Api m), Bombus pennsylvanicus (Bumble bee, Bom p), Bombus terrestris (Bumble bee, Bom t), Dolichovespula arenaria (Yellow hornet, Dol a), Dolichovespula maculata (White face hornet, Dol m), Myrmecia pilosula (Australian jumper ant, Myr p), Polistes annularis (Wasp, Pol a), Polistes dominulus (Mediterranean paper wasp, Pol d), Polistes exclamans (Wasp, Pol e), Polistes fuscatus (Wasp, Pol f), Polistes gallicus (Wasp, Pol g), Polistes metricus (Wasp, Pol m), Polybia paulista (Wasp, Pol p), Polybia scutellaris (Wasp, Pol s), Solenopsis geminata (Tropical fire ant, Sol g), Solenopsis invicta (Red imported fire ant, Sol i), Solenopsis richteri (Black fire ant, Sol r), Solenopsis saevissima (Brazilian fire ant, Sol s), Vespa crabro (European hornet, Vesp c), Vespa mandarinia (Giant asian hornet, Vesp m), Vespula fiavopilosa (Yellow jacket, Vesp f), Vespula germanica (Yellow jacket, Vesp g), Vespula maculifrons (Yellow jacket, Vesp m), Vespula pensylvanica (Yellow jacket, Vesp p), Vespula squamosa (Yellow jacket, Vesp s), Vespula vidua (Wasp, Vesp vi), Vespula vulgaris (Yellow jacket, Vesp v)), Ixodida (e.g., Argas reflexus (Pigeon tick, Arg r)), lepidoptera (e.g., Bombyx niori (Silk moth, Bomb n), Plodia interpunctella (Indianmeal moth, Plo i), Thaumetopoea pityocampa (Pine processionary moth, Tha p)), thysanura (e.g., Lepisma saccharina (Silverfish, Lep s)), Siphonaptera (e.g., Ctenocephalides felis felis (Cat flea, Cte f)), carnivora (e.g., Canis familiaris (dog, Can f), Felis domesticus (cat, Fel d)); lagomorpha (e.g., Oryctolagus cuniculus (rabbit, Ory c), Perissodactlyla: Equus caballus (domestic horse, Equ c)), Pleuronectiformes (e.g., Lepidorhombus whiffiagonis (Megrim, Whiff, Gallo, Lep w)), Rodentia (e.g., Cavia porcellus (guinea pig, Cav p), Mus musculus (mouse, Mus m), Rattus norvegius (rat, Rat n)); Coniferales: Chamaecyparis obtusa (Japanese cypress, Cha o), Cupressus arizonica (Cypress, Cup a), Cryptomeria japonica (Sugi, Cry j), Cupressus sempervirens (Common cypress, Cup s), Juniperus ashei (Mountain cedar, Jun a), Juniperus oxycedrus (Prickly juniper, Jun o), Juniperus sabinoides (Mountain cedar, Jun s), Juniperus virginiana (Eastern red cedar, Jun v)); Gentianales (e.g., Catharanthus roseus (Rosy periwinkle, Cat r)); Poales (e.g., Anthoxanthum odoratum (Sweet vernal grass, Ant o 1), Cynodon dactylon (Bermuda grass, Cyn d 1, Cyn d 7, Cyn d 12, Cyn d 15, Cyn d 22w, Cyn d 23, Cyn d 24), Dactylis glomerata (Orchard grass, Dae g 1, Dae g 2, Dae g 3, Dae g 4, Dae g 5), Festuca pratensis (Meadow fescue, Fes p 4)), Holcus lanatus (Velvet grass, Hol 1 1, Hol 1 5), Hordeum vulgare (Barley, Hor v 1, Hor v 5, Hor v 12, Hor v 15, Hor v 16, Hor v 17, Hor v 21), Lolium perenne (Rye grass, Lol p 1, Lol p 2, Lol p 3, Lol p 4, Lol p 5, Lol p 11), Oryza sativa (Rice, Ory s 1, Ory s 12), Paspalum notarum (Bahia grass, Pas n 1), Phalaris aquatica (Canary grass, Pha a 1, Pha a 5), Phleum pratense (Timothy, Phl p 1, Phl p 2, Phl p 4, Phl p 5, Phl p 6, Phl p 7, Phl p 11, Phl p 12, Phl p 13), Poa pratensis (Kentucky blue grass, Poa p 1, Poa p 5), Secale cereale (Rye, Sec c 1, Sec c 20), Sorghum halepense (Johnson grass, Sor h 1), Triticum aestivum (Wheat, Tri a 12, Tri a 14, Tri a 185, Tri a 19, Tri a 25, Tri a 26, Tri a 27, Tri a 28, Tri a 29, Tri a 30), Zea mays (Maize, Zea m 1, Zea m 12, Zea m 14, Zea m 25), Fagales: Alnus glutinosa (Alder, Aln g 1, Aln g 4), Betula verrucosa (Birch, Bet v 1, Bet v 2, Bet v 3, Bet v 4, Bet v 5, Bet v 6, Bet v 7), Carpinus betuhxs (Hornbeam, Car b 1)); Lamiales (e.g., Fraxinus excelsior (Ash, Fra e 1), Ligustrum vulgare (Privet, Lig v), Syringa vulgaris (Lilac, Syr v)); Malpighiales (e.g., Hevea brasiliensis (para rubber tree (latex), Hey b 1, Hey b 2, Hey b 3, Hey b 4, Hey b 5, Hey b 6, Hey b 7, Hey b 8, Hey b 9, Hey b 10, Hey b 11, Hey b 12, Hey b 13)); Proteales (e.g., Platanus acerifolia (London plane tree, Pla a 1, Pla a 2, Pla a 3), Platanus orientalis (Oriental plane, Pla or 1, Pla or 2, Pla or 3)).

In one embodiment, the inhaled allergen is selected from the group comprising or consisting of Acarus siro (Storage mite, Aca s 13), Dermatophagoides farinae (American house dust mite, Der f), Dermatophagoides microceras (House dust mite, Der m), Dermatophagoides pteronyssinus (European house dust mite, Der p), Euroglyphus maynei (House dust mite, Eur m), Glycyphagus domesticus (Storage mite, Gly d 2), Polistes annularis (Wasp, Pol a), Polistes dominulus (Mediterranean paper wasp, Pol d), Polistes exclamans (Wasp, Pol e), Polistes fuscatus (Wasp, Pol f), Polistes gallicus (Wasp, Pol g), Polistes metricus (Wasp, Pol m), Polybia paulista (Wasp, Pol p), Polybia scutellaris (Wasp, Pol s), Felis domesticus (cat, Fel d), Poales and Betula verrucose (Birch, Bet v 1, Bet v 2, Bet v 3, Bet v 4, Bet v 5, Bet v 6, Bet v 7).

Examples of ingested allergens include, but are not limited to, allergens from Fungi Ascomycota, such as, for example, Dothideales (e.g., Alternaria alternata (Alternaria rot fungus, Alt a), Cladosporium cladosporioides (Cla c), Cladosporium herbarum (Cla h), Curvularia lunata (Cur 1), —Eurotiales: Aspergillus flavus (Asp fl), Aspergillus fumigatus (Asp f), Aspergillus niger (Asp n), Aspergillus oryzae (Asp o), Penicillium brevicompactum (Pen b), Penicillium chrysogenum (Pen ch), Penicillium citrinum (Pen c), Penicillium oxalicum (Pen o)), Hypocreales (e.g., Fusarium culmorum (Fus c)); Onygenales (e.g., Trichophyton rubrum (Tri r), Trichophyton tonsurans (Tri t), Saccharomycetales: Candida albicans (Yeast, Cand a), Candida boidinii (Yeast, Cand b)); Tuberculariales (e.g., Epicoccum purpurascens (Epi p)), allergens from Fungi Basidiomycota, such as, for example, Hymenomycetes (e.g., Coprinus comatus (Shaggy mane, Cop c), Psilocybe cubensis (Magic mushroom, Psi c), Urediniomycetes (e.g., Rhodotorula mucilaginosa (Yeast, Rho m)); Ustilaginomycetes (e.g., Malassezia furfur (Pityriasis versicolor infect. Agent, Mala f), Malassezia sympodialis (Mala s)); antibiotics (such as, for example, Penicillins, Cephalosporins, Aminosides, Quinolones, Macrolides, Tetracycline, Sulfamids); drugs (such as, for example, acetylsalicylic acid, vaccines, morphines and derivatives); vitamins such as, for example, vitamin K1; and food allergens (such as, for example, allergen from milk, egg, peanut, tree nut (walnut, cashew, etc.), fish, shellfish, soy, wheat, and carrot, apple, pear, avocado, apricot, peach).

In one embodiment, the ingested allergen is a food allergen.

In one embodiment, the food allergen is selected from the group comprising or consisting of allergen from milk, egg, peanut, tree nut (walnut, cashew, etc.), fish, shellfish, soy, wheat, and carrot, apple, pear, avocado, apricot, peach.

Examples of contact allergens include, but are not limited to, heavy metals (such as, for example, nickel, chrome, gold), latex, haptens such as, for example halothane, hydralazine.

In one embodiment, the allergen is selected from the group comprising or consisting of Acarus siro (Storage mite, Aca s 13), Dermatophagoides farinae (American house dust mite, Der f), Dermatophagoides microceras (House dust mite, Der m), Dermatophagoides pteronyssinus (European house dust mite, Der p), Euroglyphus maynei (House dust mite, Eur m), Glycyphagus domesticus (Storage mite, Gly d 2), Polistes annularis (Wasp, Pol a), Polistes dominulus (Mediterranean paper wasp, Pol d), Polistes exclamans (Wasp, Pol e), Polistes fuscatus (Wasp, Pol f), Polistes gallicus (Wasp, Pol g), Polistes metricus (Wasp, Pol m), Polybia paulista (Wasp, Pol p), Polybia scutellaris (Wasp, Pol s), Felis domesticus (cat, Fel d), Poales and Betula verrucosa (Birch, Bet v 1, Bet v 2, Bet v 3, Bet v 4, Bet v 5, Bet v 6, Bet v 7) and food allergens.

The present invention also further relates to a method for increasing the efficacy and/or for decreasing the duration of a desensitization of a subject allergic to a specific allergen, wherein said subject is treated by desensitization, and is further administered with the immunogenic product, composition, pharmaceutical composition, medicament, or vaccine composition of the invention.

In one embodiment, in the methods of the present invention, the subject is administered first with the immunogenic product, composition, pharmaceutical composition, medicament, or vaccine composition of the invention, and second with the allergen.

In one embodiment, in the methods of the present invention, the subject is administered first with the allergen, and second with the immunogenic product, composition, pharmaceutical composition, medicament, or vaccine composition of the invention.

In another embodiment, in the method of the present invention, the subject receives a combined administration of the immunogenic product, composition, pharmaceutical composition, medicament, or vaccine composition of the invention, and of the allergen.

The present invention further relates to a composition, pharmaceutical composition, medicament or vaccine as described hereinabove, wherein said composition, pharmaceutical composition, medicament or vaccine further comprises at least one allergen.

In one embodiment, a therapeutically effective amount of at least one immunogenic product of the invention is administered or is to be administered to the subject. In one embodiment, the therapeutically effective amount corresponds to an amount of total proteins determined using a Bradford protein assay as well known in the art.

In one embodiment, the amount of the immunogenic product to be administered to the subject induces an immunoprotective response without significant adverse effects.

In one embodiment, the amount of the immunogenic product to be administered to the subject induces an allergen desensitization without significant adverse effects.

Optimal amounts of components for the immunogenic product of the invention can be ascertained by standard studies involving observation of appropriate immune responses in subjects. Following an initial vaccination, subjects can receive one or several booster immunizations adequately spaced.

In one embodiment, the treatment consists of a single dose or a plurality of doses over a period of time.

In one embodiment of the invention, the subject to be treated is administrated at least twice in a month with the therapeutically effective amount of immunogenic product as described here above.

In another embodiment of the invention, the subject to be treated is administrated twice in 1 month with a therapeutically effective amount of the immunogenic product of the invention. In this embodiment, the subject may be administrated once at day 0 and the second time between day 7 and day 28. In one embodiment, the subject is administrated once at day 0 and the second time at day 28.

In another embodiment of the invention, the subject to be treated is administrated three times in 1 month with a therapeutically effective amount of the immunogenic product of the invention. In this embodiment, the subject to be treated may be administrated once at day 0, the second time between day 7 and day 14 and the third time between day 21 and day 28. In one embodiment, the subject is administrated once at day 0, the second time at day 7 and the third time at day 28.

In another embodiment of the invention, the subject to be treated may be further administrated once every three months with the therapeutically effective amount of the immunogenic product of the invention.

In one embodiment of the invention, the subject to be treated is administered three times in one month as described here above, and then further administered once every three months with the therapeutically effective amount of the immunogenic product of the invention.

In another embodiment of the invention, the subject to be treated may be further administrated with a therapeutically effective amount of the immunogenic product as described here above when the amount of antibodies against IgE is undetectable in a serum sample obtained from the subject.

### BRIEF DESCRIPTION OF THE DRAWINGS

FIG. 1 shows the generation of hIgE Kinoid (hIgE-K). (A) Synthesis of hIgE-K using a thiol-maleimide conjugation. (B) Generation of high molecular weight kinoids upon conjugation of the IgE Cε3-Cε4 fragment to CRM₁₉₇ was confirmed using SDS-PAGE and (C) HPLC.

FIG. 2 shows the neutralization of anti-hIgE antibodies by hIgE-Kinoid. (A) Intra-muscular vaccination protocol outline. hIgE^{KI} mice (which express human IgE instead of mouse IgE) were vaccinated with hIgE-K (or CRM₁₉₇ alone as control), emulsified with the adjuvant Squalene-in-water emulsion (SWE). (B) Anti-hIgE and (C) anti-CRM₁₉₇ antibody titers in sera at 5, 9, 21, 30 and 39 weeks after first injection of kinoid. Results show values from individual mice with bars indicating medians. (D) Anti-hIgE neutralizing capacity in sera collected at week 5. Bone marrow-derived cultured mast cells (BMCMCs) expressing the hIgE receptor FcεRI were derived from mice humanized for FcεRI. BMCMCs were pre-incubated with sera from mice vaccinated with hIgE-K (collected 39 days after the first injection of vaccine) at the indicated dilution. Immediately after, fluorescently-labeled (FITC) hIgE were added for 30 min Cells were washed and levels of FITC fluorescence on BMCMCs was quantified by flow cytometry. (E) Levels of total hIgE in sera collected at week 5, 9, 21, 30 and 39. Results show values from individual mice with bars indicating mean±SEM. (B-E) Data are from a single experiment with n=8 mice per group, representative of two independent experiments. ***, P<0.001 (Mann-Whitney U test).

FIG. 3 shows that vaccination with hIgE-K prevents IgE-mediated systemic anaphylaxis. (A) Protocol outline. IgE/FcεRI humanized mice (which express human IgE and human IgE receptor FcεRI) were vaccinated (intra-muscular, i.m) with hIgE-K (or CRM₁₉₇ alone as control), emulsified with the adjuvant SWE. At week 9, mice were sensitized with hIgE anti-nitrophenyl (NP) and challenged one day later with NP (nitrophenyl) coupled to BSA both in intra-venous, as indicated. (B) Antibody titers in sera 5 weeks after first injection of kinoid. Results show values from individual mice with bars indicating medians±SEM. (C) Changes in body temperature at week 0, 1 and 3 (Δ° T, mean±SEM) after mice injection with IgE-K or CRM₁₉₇. Data are pooled from two independent experiments with a total of n=7-9 mice per group. (D) Changes in body temperature (which is used as a main readout of anaphylaxis in mice) (Δ° T, mean±SEM) after intravenous injection of 10 μg anti-NP hIgE. Data are pooled from two independent experiments with a total of n=7-9 mice per group. (E) Changes in body temperature (Δ° T, mean±SEM) after intravenous injection of 500 μg of NP-BSA. Data are pooled from two independent experiments with a total of n=7-9 mice per group. *, ** or ***, P<0.05, 0.01, or 0.001 (Mann-Whitney U test).

FIG. 4 shows that in a genetically predisposed allergic mouse model, vaccination with hIgE-K prevents IgE-mediated systemic anaphylaxis. (A) Protocol outline. IgE/FcεRI humanized mice bearing a F709 IL4Ra mutation (the equivalent mutation has been linked to atopy in human, and the mutation is known to increase susceptibility to IgE-mediated anaphylaxis in mice) were vaccinated with hIgE-K (or CRM₁₉₇ alone as control), emulsified with the adjuvant SWE. At week 6, mice were injected i.v. with 250 μg of anti-hIgE. (B) Changes in body temperature (which is used as a main readout of anaphylaxis) (Δ° T, mean±SEM) after intravenous injection of 250 μg of anti-hIgE Abs. (C) Survival curve after intravenous injection of 250 μg anti-hIgE. Data are pooled from two independent experiments with a total of n=9 mice per group. ** or ***, P<0.01, or 0.001 (Mann-Whitney U test).

### EXAMPLES

The present invention is further illustrated by the following examples.

The present invention relates to an immunogenic product using CRM₁₉₇ as a carrier protein. The properties of the immunogenic product of the invention are illustrated by the following examples.

CRM₁₉₇ is a non-toxic form of diphtheria toxin without toxic activity due to a single base substitution, in its toxin domain, from glycine to glutamate in position 52 (Uchida et al., 1973 J Biol Chem).

A thiol-maleimide conjugation is employed for the preparation of IgE based immunogenic products. Sulfhydryl moieties were introduced on the carrier protein CRM₁₉₇ with SATA and subsequent hydroxylamine deprotection, while IgE or the fragment of IgE was derivatized by sGMBS, a maleimide-containing agent. Both SATA and sGMBS are heterobifunctional crosslinkers containing a NHS-ester, which reacts with primary amines (such as ε-amino groups of lysine residues and protein N-termini).

### Example 1: Anti-IgE Vaccination Prevents Human IgE-Mediated Severe Allergic Reactions in Humanized Mice

### Materials and Methods

### Mice

hIgE^{KI} mice were obtained inserting human IgE sequence (1080 base pair, located on human chromosome 14: 106,064,224-106,068,065) on mouse chromosome 12 (Chr12:113,147,778). IgE/FcεRI humanized mice were generated by intercrossing of hIgE^{KI} and mFcεRI^{-/-} hFcεRI^{Tg} mice (Dombrowicz D et al., Anaphylaxis mediated through a humanized high affinity IgE receptor. Journal of immunology (Baltimore, Md: 1950). 1996; 157(4):1645-51). IgE/FcεRI humanized mice bearing the F709 IL4Ra mutation were generated by intercrossing of IgE/FcεRI humanized mice with F709 IL4Ra mice (Tachdjian R et al., In vivo regulation of the allergic response by the IL-4 receptor alpha chain immunoreceptor tyrosine-based inhibitory motif. J Allergy Clin Immunol. 2010; 125(5):1128-36.e8). Mice were maintained in a specific pathogen-free facility at Institut Pasteur. Mice were bred at Institut Pasteur and demonstrated normal development and breeding patterns. All animal care and experimentation were conducted in compliance with the guidelines and specific approval of the Animal Ethics committee CETEA (Institut Pasteur, Paris, France) registered under #170043, and by the French Ministry of Research.

### IgE Fragments Production

The recombinant hIgE Cε3-4 fragment (containing G335C mutation, with C-terminal Strep Twin tag and harboring the amino acid sequence of SEQ ID NO: 7) was synthesized and transiently transfected into exponentially growing Expi-293 cells that were cultured in Expi293™ Expression Medium (Life Technologies) in suspension at 37° C. in a humidified 5% CO2 incubator on a shaker platform rotating at 110 rpm. Twenty-four hours before transfection, cells were harvested resuspended in Expi293™ Expression Medium at a density of 2×10⁶ cells/ml, and cultured overnight in the same conditions as mentioned above. Twenty-four hours after, 500 μg of expressing plasmids and 1350 μL of Expifectamine were pre-incubated during 5 min in Opti-MEM (Life Technologies) medium and mixed together. After 20 minutes of incubation, the mixture is added to Expi-293 cells at density of 2.9×10⁶ cells/mL. Twenty hours after the transfection, 25 mL and 2.5 mL of transfection enhancer 1 and 2 (ThermoFisher) respectively were added. Cells were cultured for 5 days after transfection, supernatants were harvested, centrifuged at 4200 rpm for 30 min and filtered (0.2 μm). Proteins were purified by affinity chromatography using an AKTA pure FPLC instrument (GE Healthcare) and Strep-Tactin® Column (IBA Lifescience).

Synthesis and Characterization of hIgE Kinoid

hIgE Cε3-4 was modified with N-γ-maleimidobutyryl-oxysuccinimide ester (sGMBS; Thermo Fisher), a maleimide-containing agent reacting with primary amines. Buffer of hIgE Cε3-4 was exchanged against modification buffer (70 mM Phosphate buffer, 150 mM NaCl, 5 mM EDTA, pH=7.2) at 1 mg/mL. A solution of 10 mM of sGMBS was prepared and added to the hIgE Cε3-4 at a 1:30 ratio and incubated during 60 minutes at room temperature (protected from light). Excess sGMBS was removed and buffer exchanged against modification buffer using Zeba desalting spin column (Thermo Fisher). CRM₁₉₇ was purchased from Pfenex (USA). Sulfhydryl moieties were introduced on the carrier protein CRM₁₉₇ with SATA (N-succinimidyl-S-acetylthioacetate.). CRM₁₉₇ was diluted in modification buffer at 2 mg/mL and a freshly prepared solution of 100 mM SATA (dissolved in DMSO) was added at a 1:80 molar ratio and incubated 30 minutes at room temperature (protected from light). Excess SATA was removed and buffer exchanged against modification buffer using Zeba desalting spin column SATA modified CRM₁₉₇ was incubated with a solution of hydroxylamine at a 50 mM final concentration, at room temperature for 120 minutes, protected from light. Excess hydroxylamine was removed and buffer exchanged against modification buffer using Zeba desalting spin column After CRM₁₉₇ and hIgE Cε3-4 functionalization, protein content of each preparation was determined by Bradford (Thermo Fisher) assay according to manufacturer's instructions.

Functionalized CRM₁₉₇ was added to functionalized hIgE Cε3-4 at a molar ratio of 1:1 and a final concentration of 0.4 mg/mL. The mixture was incubated 16 hours at 4° C., protected from light, and subsequently buffer exchanged against modification buffer using Zeba desalting spin column. Protein content was determined by Bradford assay. Resulting hIgE kinoid (hIgE-K) was then 0.22 μm sterile filtered and stored at 4° C.

The hIgE-K was characterized using different in vitro methods. To analyze the profiles of the kinoids obtained, SDS-PAGE and western blot were performed against the hIgE Cε3-4 fragment (Strep-TACTIN HRP conjugate (IBA Lifescience)). Size exclusion (SE)-HPLC using a Bio SEC-5 column (2000 Å, 5 μm, 7.8*300 mm, Agilent) and Bio SEC-3 column (300 Å, 3 μm, 7.8*300 mm, Agilent) was also used. SE-HPLC analysis were performed in the isocratic mode at 1 mL/min with column temperature at 25° C. After filtration (0.22 μm-cut-off), samples were injected at 100 μL and analyzed at 280 nm. The total run time was 35 min.

### Production of Human IgE Antibodies

Anti-nitrophenyl hIgE were produced and purified as described previously (B albino B et al., The anti-IgE mAb omalizumab induces adverse reactions by engaging Fcgamma receptors. J Clin Invest. 2020). JW8/5/13 (ECACC 87080706) cells were obtained from Sigma-Aldrich. This cell line produces a chimeric human IgE antibody directed against the hapten 4-hydroxy-3-nitrophenacetyl (NP), and composed of the human Fcε chain and mouse anti-NP variable chain. JW8/5/13 cells were cultured in complete Dulbecco-modified Eagle medium (DMEM, Gibco) containing 2 mM glutamine (Thermo Fisher Scientific) and 10% Fetal Bovine Serum (FBS) (Thermo Fisher Scientific) at 9×10⁵ cells/ml. After 15 days, supernatants were harvested, centrifuged at 4200 rpm for 30 min and filtered (0.2 μm). We purified IgE antibodies by affinity chromatography. Briefly, CNBr-activated Sepharose 4 Fast Flow Beads (GE Healthcare) were coupled with WT anti-IgE using a ratio of 2.5 mg of protein for each gram of beads. Beads were weighted, washed with 15 volumes of cold 1 mM HCl and centrifuged for 5 min at 2500 rpm. WT anti-IgE were resuspended in coupling solution (0.1 M NaHCO3 pH 8.3 containing 0.5M NaCl) and mixed with beads overnight at 4° C. under agitation. Beads were washed with coupling buffer and non-reacted groups were blocked with 0.1 M Tris-HCl buffer pH 8.0. WT anti-IgE-coupled beads were then washed using alternate low (0.1 M acetate buffer pH 3) and high (0.1 M Tris-HCl pH 8) pH solutions and stored in Borate buffer (100 mM Borate, 150 mM NaCl pH 8.0) at 4° C. until use. For purification of IgE, WT anti-IgE-coupled sepharose beads were packed in XK 16/20 Column (GE Healthcare) and affinity chromatography was performed using an AKTA pure FPLC instrument (GE Healthcare). After purification, IgE antibodies were desalted with HiTrap Desalting Column (GE Healthcare), and stored at 4° C. until use.

Vaccination with hIgE Kinoid

Mice were immunized intramuscularly with hIgE-K combined 1:1 (v:v) with SWE, a squalene-in-water emulsion adjuvant (Vaccine Formulation Laboratory, University of Lausanne, Switzerland) in PBS at day 0, 7 and 28 with two initial doses of 30 μg followed by a boost of 10 μg. As controls, groups of mice were injected with CRM₁₉₇ following the same schedule with two initial doses of 15 μg followed by a boost of 5 μg (these doses were defined based on the weight ratio of CRM₁₉₇) combined with SWE.

Quantification of IgG Against Human IgE and CRM₁₉₇ in Sera from Vaccinated Mice

The immunogenicity of the kinoid was assessed by evaluating antibodies against human IgE and CRM₁₉₇ in sera collected at different time points after vaccination. Human IgE or CRM₁₉₇ were coated at 4° C. at 5 or 1 μg/mL respectively in coating buffer (carbonate/bicarbonate buffer pH 9.6) and incubated overnight. After each step, plates were washed three times with PBS Tween 20 at 0.005%. After blocking with BSA 1% PBS, serum samples were added, a two-fold serial dilution was conducted starting at 2000 dil⁻¹ (diluted in PBS, BSA 1%). After 90 minutes of incubation at 37° C., bound antibodies were detected with HRP-conjugated goat anti mouse IgG (Bethyl Laboratories) at 1/10 000 and plates were revealed using an OPD substrate. Reaction was stopped with 1 M H₂SO₄ and absorbance was subsequently recorded at 490 nm. Samples were analyzed starting at dilution 2000 dil⁻¹ up to 1 024 000 dil⁻¹. The titers were defined as the dilution of the serum where 50% of the OD max. Titers were expressed as serum dilution factors (dil⁻¹). The limit of titer quantification is the lowest dilution tested in the assay: 2000 dil⁻¹.

Assessment of the Neutralizing Capacities of Anti-hIgE Antibodies Produced Upon Vaccination with hIgE-K

Bone marrow-derived cultured mast cells (BMCMCs) expressing hFcεRI were obtained by culturing bone marrow cells from IgE/FcεRI humanized mice in medium containing IL-3 (10 ng/ml) for 6 weeks, at which time cells were >95% c-Kit⁺hFcεRIα⁺ (data not shown). To assess the neutralizing capacity of anti-hIgE antibodies produced upon vaccination with hIgE-K, we incubated BMCMCs with dilutions of plasma from mice vaccinated with hIgE-K or CRM₁₉₇ alone (as a control). We then added FITC-labeled hIgE (produced as described previously in B albino B et al., The anti-IgE mAb omalizumab induces adverse reactions by engaging Fcgamma receptors. J Clin Invest. 2020), and assess binding of FITC-hIgE to hFcεRI on BCMMCs by flow cytometry.

### IgE Quantification on the Surface of Basophils and Mast Cells

Blood was collected with heparin. For peritoneal lavage fluid (PLF), the outer skin of the peritoneum was gently removed. Then 3 mL of cold PBS was injected into the peritoneal cavity using a 27 g needle. After a gently massage of the peritoneum, an incision was performed in the inner skin of the peritoneum and while holding up the skin with forceps, the PLF was recovered.

### Red Blood Cell Lysis was Carried Out to Remove Red Blood Cells

Cells coming from blood were stained with anti CD49b-BV421 (clone DXS, eBioscience), anti CD131-PE (clone REA193, Miltenyi) and with anti-human IgE-biotin (clone MHE-18, Biolegend) and anti-Biotin-APC (clone REA746, Miltenyi) or anti-human FcεRI-APC (clone AER-37 (CRA-1), BioLegend). Cells coming from PLF were stained with anti cKIT-APC (clone 2B8, eBioscience) and with anti-human IgE biotin (clone MHE-18, BioLegend) and anti-Biotin-APC (clone REA746, Miltenyi) or anti-human FcεRI-APC (clone AER-37 (CRA-1), BioLegend). Basophils were gated as CD49b⁺ CD131⁺ and mast cells as cKIT⁺IgE⁺ or FcεRI⁺. Surface expression of human FcεRI and IgE was assessed and expressed by mean fluorescence intensity (MFI).

### Total Human and Mouse IgE Quantification

Total human IgE levels were quantified by ELISA. Anti-Cε2 human IgE antibody (clone 8E/5D4, Aviva Systems Biology) was coated and incubated overnight at 4° C. at 5 μg/mL in coupling buffer (carbonate/bicarbonate buffer pH=9.6). After each step, plates were washed three times with PBS Tween 20 at 0.005%. After blocking with BSA 1% in PBS for 1 h30 at room temperature, serum samples were added at 1/10 final dilution (diluted in PBS, BSA 1% 10% FBS) and incubated for 90 minutes at room temperature. Then, anti-human IgE antibody (A80-108P, Bethyl Laboratories) were added at 1:10,000 during 90 minutes at room temperature. Plates were revealed using OPD substrate. Reaction was stopped with 2 M H₂SO₄ and absorbance was subsequently recorded at 490 nm. Total mouse IgE levels were quantified by ELISA using a commercial ELISA kit (E90-115; B ethyl Laboratories) according to the manufacturer's instructions.

### Passive Systemic Anaphylaxis

In IgE/FcεRI humanized mice, purified mouse IgE anti-NP antibodies were administered intravenously (i.v.) at a dose of 10 μg in 100 μL of PBS. Twenty-four hours later, mice were challenged i.v. with 500 μg of NP (21-31)-BSA (Santa Cruz Biotechnology) in PBS. Rectal measurements of body temperature were performed immediately before (time 0) and at different time points for up to one hour after challenge. In IgE/FcεRI humanized; F709 IL4Ra mice, rabbit anti-hIgE antibodies (Bethyl Laboratories) were administered i.v. at a dose of 250 μg. Rectal measurements of body temperature were performed immediately before (time 0) and at different time points for up to one hour after the injection.

### Statistical Analysis

Statistical significance was determined using the unpaired Student's t test (unpaired Mann Whitney test). P≤0.05 was considered statistically significant. Calculations were performed using the Prism 7.0 software program (GraphPad Software).

### Results

Vaccination with hIgE Kinoid Induces Potent Anti-IgE Neutralizing Antibodies in IgE Humanized Mice

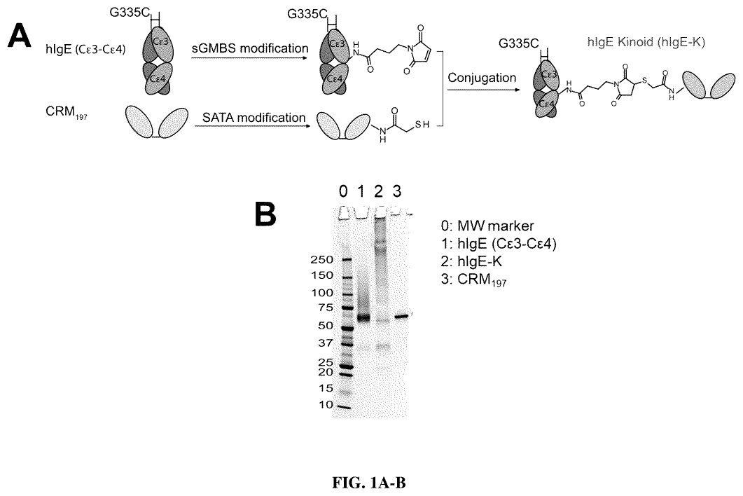

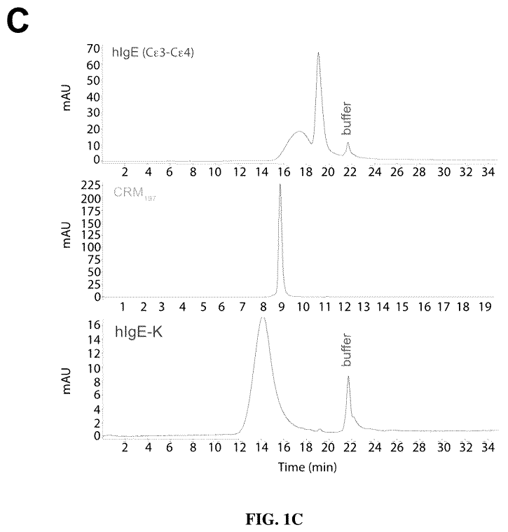

IgE kinoids (hIgE-K) were generated by coupling human IgE Cε3-4 domains with diphtheria ‘cross-reactive material ₁₉₇’ (CRM₁₉₇, a non-toxic mutant of diphtheria toxin used as a carrier protein in a number of approved conjugated vaccines) using a thiol-maleimide conjugation (FIG. 1A). We replaced the native glycine residue at position 335 by a cysteine residue into Cε3-4. Consequently, interchain disulfide bonds are formed that locks the IgE fragment into a “closed” conformation retaining high-affinity binding to omalizumab, but not FcεRI. We hypothesized that an IgE conjugate vaccine containing this G335C mutation would favor generation of “omalizumab-like” neutralizing antibodies while avoiding potentially harmful binding to FcεRI. SDS-PAGE and HPLC analysis indicated formation of high molecular species upon conjugation of hIgE Cε3-4 G335C to CRM₁₉₇, confirming efficiency synthesis of hIgE-K (FIGS. 1B and 1C).

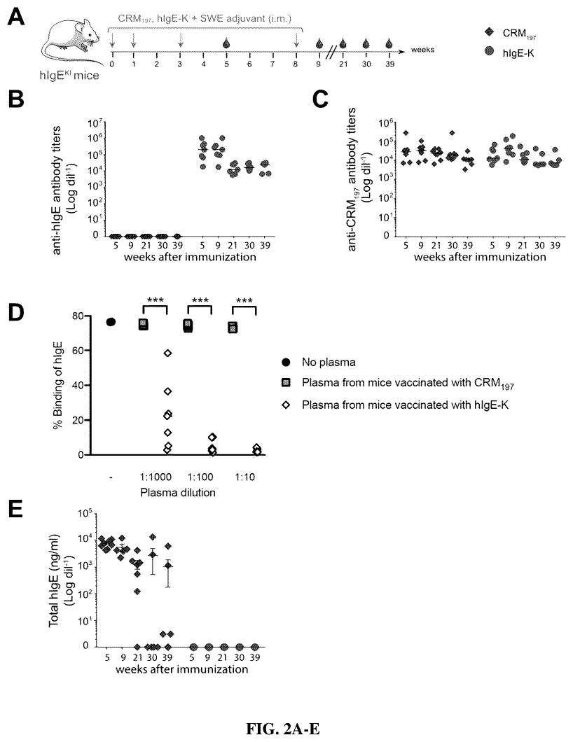

Immunization of hIgE^{KI} mice (which express human IgE instead of mouse IgE) with hIgE-K in SWE, a squalene oil-in-water emulsion adjuvant, induced high anti-hIgE antibody titers, detectable already 5 weeks after primary immunization and still more than 39 weeks after (the latest time-point assessed so far) (FIG. 2A-B). As expected, all mice exposed to CRM₁₉₇ alone or hIgE-K developed anti-CRM₁₉₇ antibodies (FIG. 2C). Importantly, anti-hIgE antibodies generated upon vaccination with the kinoid exhibited strong neutralizing capacities in all mice starting 5 weeks after primary immunization (FIG. 2D). We could detect hIgE in the blood of CRM₁₉₇-immunized control mice, but not in hIgE^{KI} mice vaccinated with the hIgE-K, confirming the neutralizing capacities of antibodies generated upon vaccination (FIG. 2E). Altogether, these data indicate that efficient long-term neutralization of hIgE can be achieved through vaccination with hIgE-K in hIgE^{KI} mice.

Efficacy of Anti-hIgE Vaccine in a Model of hIgE-Mediated Anaphylaxis

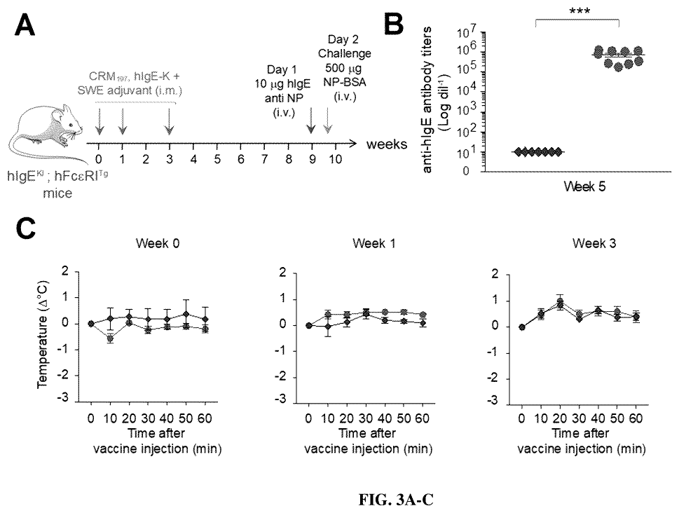

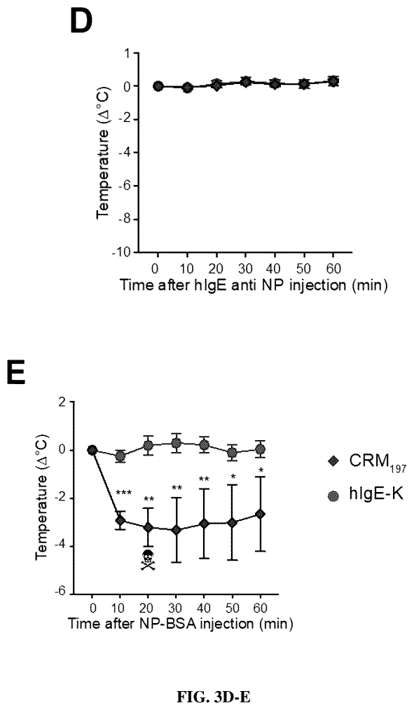

We assessed potential adverse events following injection of hIgE-K in mice expressing both human IgE and human FcεRI (IgE/FcεRI humanized mice). We carefully monitored mice after each injection of IgE-K vaccine (or CRM₁₉₇ alone as a control) (FIG. 3A) and did not observe any detectable adverse effect in IgE/FcεRI humanized mice: neither hypothermia (FIG. 3C), the parameter used to follow anaphylactic shock in mice, nor diarrhea, distress or lack of vitality over 1 hour following each vaccine injection in IgE/FcεRI humanized mice. This absence of adverse effects suggests that the vaccine does not trigger FcεRI activation through IgE aggregation on the surface of mast cells and basophils. Vaccination with hIgE-K induced high titers of anti-hIgE antibodies in IgE/FcεRI humanized mice, which were already detectable 5 weeks after the first injection of kinoid (FIG. 3B), similarly to their appearance in hIgE^{KI} mice (FIG. 2E).

To further assess the safety and efficiency of the hIgE vaccine, we injected a high dose (10 μg) of anti-nitrophenyl (NP) hIgE into IgE/FcεRI humanized mice which had been vaccinated with hIgE-K or with CRM₁₉₇ alone as a control, following the same immunization schedule described above (FIG. 3A). Again, we observed neither hypothermia, nor diarrhea, distress or lack of vitality over 1 hour following injection of anti-NP-hIgE, confirming that the vaccine does not induce detectable side effects even in the presence of very high levels of circulating hIgE (FIG. 3D). Importantly, mice vaccinated with the hIgE-K were protected from hIgE-mediated anaphylaxis, whereas CRM₁₉₇-vaccinated mice injected with anti-NP hIgE and challenged with the NP antigen suffered profound hypothermia and 1 out of 7 mice died (FIG. 3E).

### Efficacy of Anti-hIgE Vaccine in a Genetically Predisposed Allergic Mouse Model

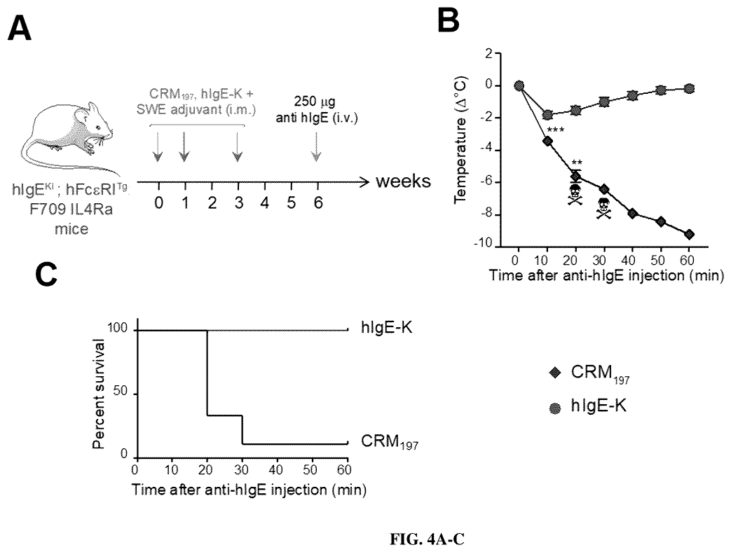

IgE/FcεRI humanized mice demonstrated low levels of circulating hIgE, whereas allergic patients display moderate to high levels of circulating IgE, making the mouse model potentially easier to protect from IgE-induced events following anti-IgE vaccination. To resolve this discrepancy, we crossed IgE/FcεRI humanized mice with mice bearing the gain-of-function Y709F mutation in the gene encoding the interleukin-4 (IL-4) and IL-13 receptor subunit, IL-4Ra, to generate hIgE^{KI}; hFcεRI^{Tg}; F709 IL4Ra mice (FIG. 4A). The Y709F mutation disrupts the Immunoreceptor tyrosine-based inhibitory motif (ITIM) of IL4Ra, thus enhancing receptor signaling in response to IL-4 and IL-13, amplifying IgE levels and IgE-mediated anaphylaxis. We used hIgE^{KI}; hFcεRI^{Tg}; F709 IL4Ra mice to assess the efficiency of the hIgE vaccine in a model in which anaphylaxis is triggered by endogenous hIgE. To do so, we injected hIgE^{KI}; hFcεRI^{Tg}; F709 IL4Ra mice with a high dose of polyclonal anti-hIgE Abs to trigger mast cell activation through crosslink of FcεRI-bound hIgE. CRM₁₉₇-immunized mice, used as controls, developed severe hypothermia (FIG. 4B) with 100% mortality within 30 min after anti-hIgE injection (FIG. 4C), confirming that IgE/FcεRI humanized; F709 IL4Ra mice have sufficient levels of endogenous hIgE bound to hFcεRI to trigger hIgE-mediated anaphylaxis. By great contrast, after anti-hIgE injection, IgE-K vaccinated mice displayed only a transient mild hypothermia and suffered no mortality (FIG. 4B-C).

Altogether, our results indicate that a vaccine against human IgE Cε3-4 domains can be produced using standard industrial methods, and this vaccine can lead to long-term neutralization of hIgE leading to undetectable IgE levels in circulation and reduced FcεRI-bound hIgE. hIgE-K vaccination does not induce any detectable adverse effects in mice humanized for IgE and FcεRI, even after repeated injections. IgE-K vaccination leads to protection from severe IgE-mediated allergic reactions, even in genetically predisposed allergic mouse models. These results pave the way for the clinical development of an efficient long-term vaccine against hIgE-mediated allergic disorders.

1-16. (canceled)

17. An immunogenic product comprising at least one immunoglobulin or immunoglobulin fragment conjugated with a carrier protein, wherein the at least one immunoglobulin is IgE, and wherein the IgE fragment comprises the IgE Cε3 domain.

18. The immunogenic product according to claim 17, wherein the carrier protein is CRM₁₉₇.

19. The immunogenic product according to claim 17, wherein the immunoglobulin fragment comprises a part or the totality of the IgE Cε3 and Cε4 domains.

20. The immunogenic product according to claim 17, wherein the immunoglobulin fragment comprises a part or the totality of the IgE Cε2, Cε3 and Cε4 domains.

21. The immunogenic product according to claim 17, wherein the IgE or the fragment thereof comprises the G335C mutation.

22. The immunogenic product according to claim 17, wherein the IgE fragment comprises or consists in SEQ ID NO:7.

23. The immunogenic product according to claim 17, wherein the IgE or the IgE fragment comprises at least one glycosylation.

24. A composition comprising the immunogenic product according to claim 17.

25. The composition according to claim 24, being a pharmaceutical composition and comprising at least one pharmaceutically acceptable excipient.

26. The composition according to claim 24, being a vaccine composition and comprising at least one adjuvant.

27. The composition according to claim 24, being an emulsion.

28. A method for producing an immunogenic product according to claim 17, the method comprising steps of:
(a) contacting the immunoglobulin or fragment thereof with a heterobifunctional crosslinker containing a NHS-ester, thereby obtaining a complex between a heterobifunctional crosslinker containing a NHS-ester and the immunoglobulin or fragment thereof;
(b) contacting the carrier protein with a heterobifunctional crosslinker containing a NHS-ester to generate a complex between the heterobifunctional crosslinker containing a NHS-ester and the carrier; and
(c) contacting the complex between a heterobifunctional crosslinker containing a NHS-ester and the immunoglobulin or fragment thereof obtained at step (a) with the complex between the heterobifunctional crosslinker containing a NHS-ester and the carrier obtained at step (b).

29. The method according to claim 28, the method comprising steps of:
(a) contacting the immunoglobulin or fragment thereof with N-[γ-maleimidobutyryloxy]-succinimide ester (sGMBS), thereby obtaining a sGMBS-immunoglobulin or fragment thereof complex;
(b) contacting the carrier protein with N-succinimidyl-S-acetylthioacetate (SATA) to generate a carrier-SATA complex; and
(c) contacting the sGMBS-immunoglobulin or fragment thereof complex obtained at step (a) with the carrier-SATA complex obtained at step (b).

30. A method for treating an inflammatory disorder in a subject, comprising administering to the subject an immunogenic product according to claim 17.

31. The method according to claim 30, wherein the inflammatory disorder is associated with aberrant IgE expression or activity.

32. The method according to claim 30, wherein the inflammatory disorder is selected from the group comprising asthma, allergic conditions, anaphylaxis, atopic disorders, bullous pemphigoid, respiratory disorders, nasal polyposis and other conditions involving airway inflammation; inflammatory and/or autoimmune disorders or conditions, gastrointestinal disorders or conditions; systemic lupus erythematosus; mastocytosis and mast cell activation syndrome (MCAS).

33. The method according to claim 30, wherein the inflammatory disorder is selected from the group comprising food allergies, venom allergy, allergy to animals, drug allergy, hyper IgE syndrome, allergic rhinitis, allergic conjunctivitis and allergic enterogastritis, urticaria, eczema, asthma, allergic bronchopulmonary aspergillosis, allergic bronchopulmonary mycosis, eosinophilia, fibrosis and excess mucus production systemic sclerosis inflammatory bowel diseases (IBD), eosinophilic esophagitis (EE), eosinophilic-mediated gastrointestinal disease, ulcerative colitis and Crohn's disease.

34. The method according to claim 30, wherein the inflammatory disorder is selected from allergy, anaphylaxis, allergic asthma, allergic rhinitis, allergic conjunctivitis, nasal polyposis.

35. The method according to claim 30, wherein the inflammatory disorder is food or venom allergy.

36. The method according to claim 30, wherein the method is for inducing desensitization of a subject allergic to a specific antigen, wherein said immunogenic product or composition and said specific antigen are administered to the allergic subject.
